(19) United States
(12) Patent Application Publication
(10) Pub. No.: US 2025/0276968 A1
(43) Pub. Date: Sep. 4, 2025

(54) 8- AND 6-SUBSTITUTED PYRIDOPYRIMIDINE DERIVATIVES AS KRAS INHIBITORS

(71) Applicant: Beta Pharma, Inc., Wilmington, DE (US)

(72) Inventors: Don Zhang, Princeton, NJ (US); Jirong Peng, Meqoun, WI (US); Michael John Costanzo, Bonney Lake, WA (US); Michael Alan Green, Easton, PA (US); Michael Nicholas Greco, Lansdale, PA (US); Stephen Bolgunas, Swedesboro, NJ (US)

(21) Appl. No.: 18/859,365

(22) PCT Filed: May 8, 2023
(86) PCT No.: PCT/US2023/021337

§ 371 Date: Oct. 23, 2024

Related U.S. Application Data
(60) Provisional application No. 63/339,580, filed on May 9, 2022.

(51) Int. Cl. C07D 471/04; A61K 31/519

(52) U.S. Cl. CPC C07D 471/04; A61K 31/519

(57) ABSTRACT

The present invention is directed to inhibitors of Kirsten Rat sarcoma virus (KRAS), and more particularly to compounds of Formula (I), as well as compositions comprising Formula (I) and methods of using the compound of Formula (I) for the treatment or prevention of a disease, disorder, or medical condition mediated through KRAS, especially the KRAS mutant G12C.

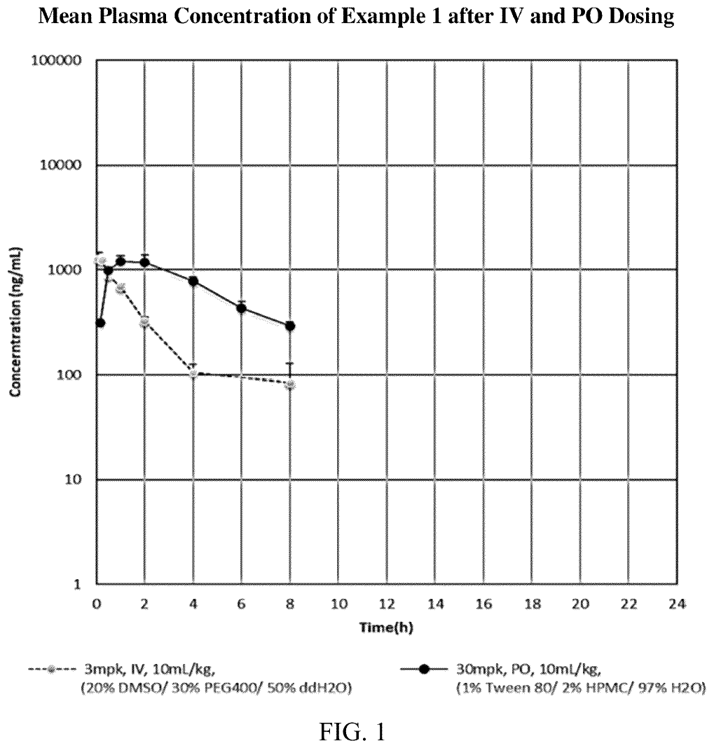

### CROSS REFERENCE TO RELATED APPLICATIONS

This application claims priority under 35. U.S.C. § 119 (c) to U.S. Provisional Patent Application No. 63/339,580, filed on May 9, 2022, and which is incorporate by reference in its entirety.

### BACKGROUND OF THE INVENTION

### Field of the Invention

The present invention is directed to inhibitors of Kirsten Rat sarcoma virus (KRAS), and more particularly to pyridopyrimidine compounds, compositions and methods for the treatment or prevention of a disease, disorder, or medical condition mediated through KRAS, especially the KRAS mutant G12C. The diseases include various cancers.

### Brief Description of the Related Art

Ras is a superfamily of small guanosine triphosphate (GTP) binding proteins consisting of various isoforms. Ras genes can mutate to oncogenes that are associated with numerous cancers such as lung, pancreas, and colon. Ras is one of the most frequently mutated oncogenes. KRAS, (Kirsten Rat sarcoma virus) an isoform of Ras, is one of the most frequently mutated Ras genes, comprising approximately 86% of all mutations. KRAS functions as an on/off switch in cell signaling. KRAS is a proto-oncogene that operates between inactive (GDP-bound) and active (GTP-bound) states to control a variety of functions, including cell proliferation. However, KRAS mutation leads to uncontrolled cell proliferation and cancer. KRAS-4B is the major isoform in cancers of the colon (30-40%), lung (15-20%) and pancreas (90%). (Liu, P. 2019, Acta Pharmaceutica Sinica B). Consequently, inhibitors of KRAS-GTP binding represent potential therapeutic agents for the treatment of various cancers.

Past attempts to design KRAS inhibitors have been mostly unsuccessful, due in large part to the high affinity of KRAS for GTP. However, more recent approaches that target the KRAS G12C mutation have shown more promise. This mutation exists in roughly 50% of lung cancers and approximately 10-20% of all KRAS G12 mutations. The cysteine residue of the mutation is positioned within the active site such that the sulfhydryl functionality can form a covalent bond with a suitably functionalized bound ligand (Liu, P. 2019, Acta Pharmaceutica Sinica B). This approach has identified irreversible, covalent inhibitors of the KRAS G12C mutation that are undergoing clinical study. Given the prominent role that KRAS plays as a driver of many malignancies, a need for new KRAS inhibitors with improved selectivity, safety, and efficacy exists.

### SUMMARY OF THE INVENTION

In one aspect, the present invention is directed to a compound of Formula I:

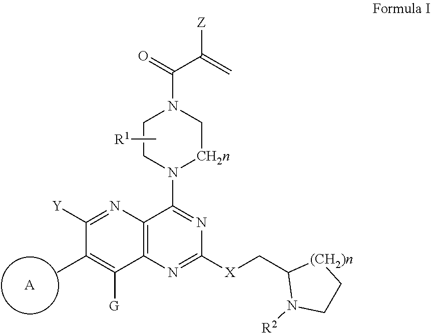

or a salt, solvate, or prodrug thereof, wherein
   A is chosen from aryl or heteroaryl optionally substituted with one or more of hydrogen, halogen, hydroxyl, C₁₋₆alkyl, C₂-C₆alkenyl, C₂-C₆alkynyl, —(C₀-C₆alkyl)cycloalkyl, C₁₋₆haloalkyl, C₁₋₆alkoxy, NO₂, cyano, CO₂H, PO(OR³)₂, POR³(OR³), PO(R⁴)₂, NH₂, NH(C₁₋₆ alkyl) or N(C₁₋₆ alkyl)₂;
   X is chosen from O, NR², S or CH₂;
   Y and G may be the same or different and chosen from bromine, iodine, C₁₋₄alkyl, C₁₋₄ perdeuteroalkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl);
   Z is chosen from hydrogen, halogen, trifluoromethyl or C₁₋₆ alkyl;
   R¹ is chosen from hydrogen, C₁-C₆alkyl, —(C₁-C₆alkyl)C₁₋₆alkoxy, —C₀-C₆alkyl(cycloalkyl), C₁-C₆haloalkyl, —(C₁-C₆alkyl)CN or —(C₁-C₆alkyl)P(O)R²R³;
   n is 1-3;
   Each R² is chosen from H, C₁₋₆ alkyl, C₃₋₆cycloalkyl or —(C₁-C₆alkyl)P(O)R²R³;
   R³ is chosen from H, C₁₋₆alkyl or C₃₋₆cycloalkyl;
   R⁴ is chosen from C₁₋₆ alkyl, C₃₋₆cycloalkyl or aryl.

In another aspect, the present invention is directed to a pharmaceutical composition comprising a compound or salt of Formula I together with a pharmaceutically acceptable carrier.

In another aspect, the present invention is directed to a method of treating a disease, disorder, or medical condition in a patient, comprising the step of providing to a patient in need thereof a therapeutic agent, wherein the therapeutic agent comprises the compound of Formula I or salt thereof.

These and other aspects will become apparent upon reading the following detailed description of the invention.

### BRIEF DESCRIPTION OF THE DRAWINGS

The FIGURE is a graph showing the mean plasma concentration of Example 1 after IV and PO dosing.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

All Formulae disclosed herein include all pharmaceutically acceptable salts of such Formulae.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₈alkyl, C₁-C₄alkyl, and C₁-C₂alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Peptide” means a molecule which is a chain of amino acids linked together via amide bonds (also called peptide bonds).

“Pharmaceutical compositions” means compositions comprising at least one active agent, such as a compound or salt of Formula II, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of the Formulae disclosed herein may contain one or more asymmetric elements such as stereogenic centers, stereogenic axes, and the like, e.g., asymmetric carbon atoms, so that the compounds can exist in different stereoisomeric forms. These compounds can be, for example, racemates, atropisomers, or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates and atropisomers can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, atropisomeric forms, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stercochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

“Salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca. Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts. In an embodiment, the compounds of the present invention are synthesized or isolated as trifluoroacetic acid (TFA) salts.

In one embodiment, the salt forms of the compounds of the present invention described above may include pharmaceutically acceptable salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

The compounds of the present invention relate to substituted pyridopyrimidine derivatives or a pharmaceutically acceptable salts, solvates, or prodrugs thereof, wherein the 4-amino group contains a functionality such as but-3-ene-2-one, as shown in Formula I:

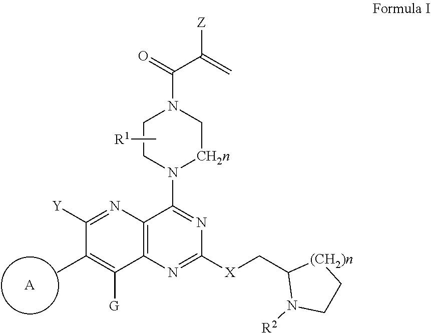

In Formula I, A is chosen from aryl or heteroaryl optionally substituted with one or more of hydrogen, halogen, hydroxyl, C₁₋₆alkyl, C₂-C₆alkenyl, C₂-C₆alkynyl, —(C₀-C₆alkyl)cycloalkyl, C₁₋₆haloalkyl, C₁₋₆alkoxy, NO₂, cyano, CO₂H, PO(OR³)₂, POR³(OR³), PO(R⁴)₂, NH₂, NH(C₁₋₆ alkyl) or N(C₁₋₆ alkyl)₂;
X is chosen from O, NR², S or CH₂;
Y and G may be the same or different and chosen from bromine, iodine, C₁₋₄alkyl, C₁₋₄ perdeuteroalkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl);
Z is chosen from hydrogen, halogen, trifluoromethyl or C₁₋₆ alkyl;
R¹ is chosen from hydrogen, C₁-C₆alkyl, —(C₁-C₆alkyl)C₁₋₆alkoxy, —C₀-C₆alkyl(cycloalkyl), C₁-C₆haloalkyl, —(C₁-C₆alkyl)CN or —(C₁-C₆alkyl)P(O)R²R³;
n is 1-3;
Each R² is chosen from H, C₁₋₆ alkyl, C₃₋₆cycloalkyl or —(C₁-C₆alkyl)P(O)R²R³;
R³ is chosen from H, C₁₋₆alkyl or C₃₋₆cycloalkyl;
R⁴ is chosen from C₁₋₆ alkyl, C₃₋₆cycloalkyl or aryl.

In the preferred embodiments, the compounds of Formula I are represented by 1a-1z and 2a-2g or a pharmaceutically acceptable salt, solvate, or prodrug thereof:

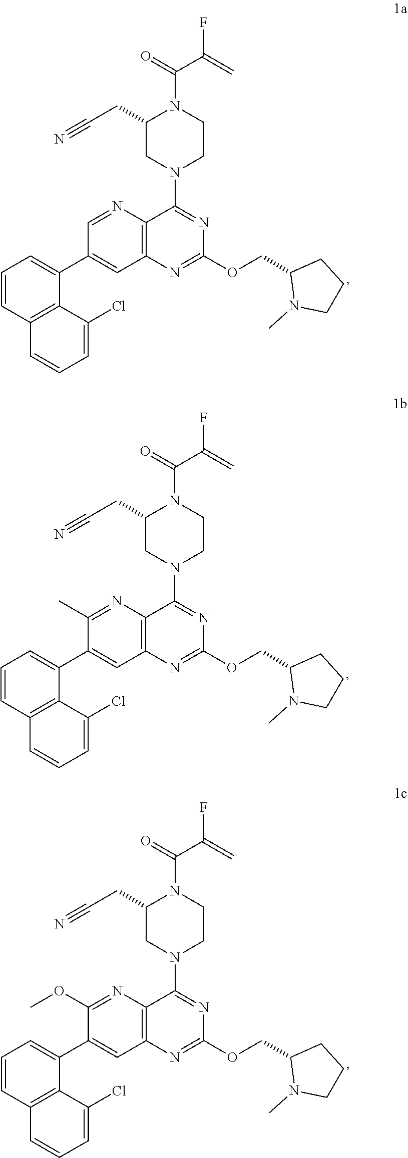

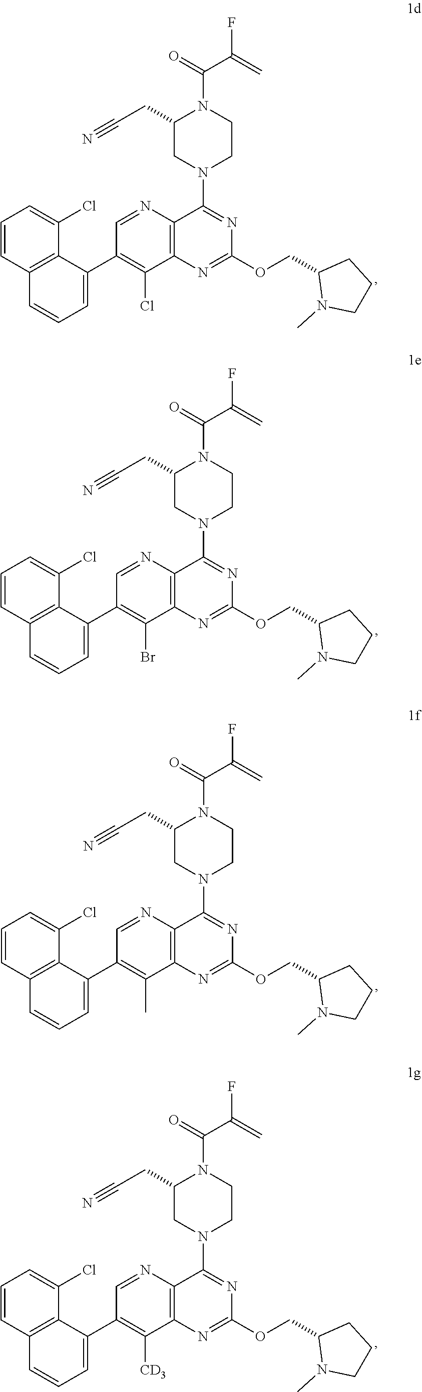

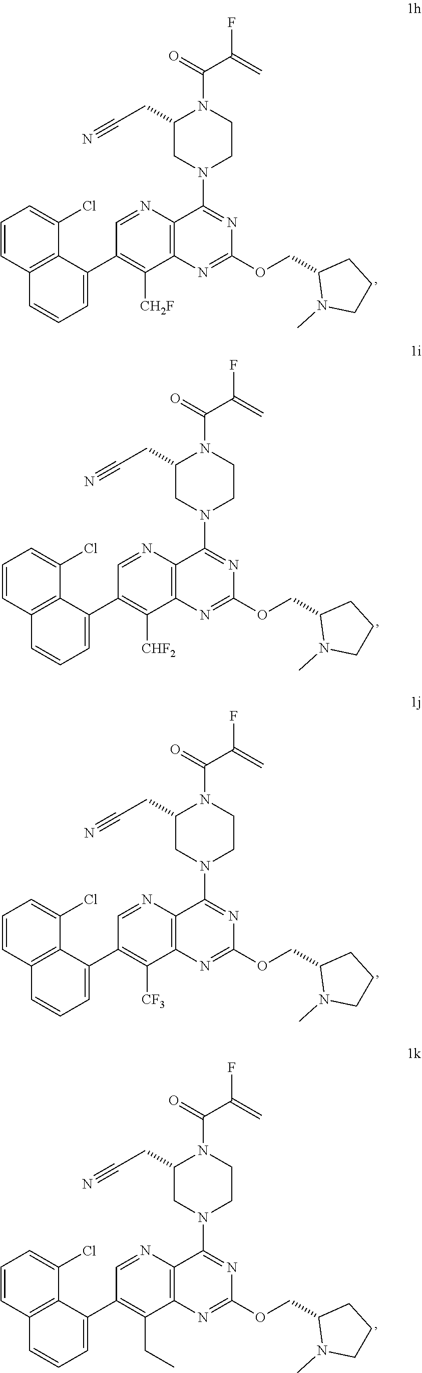

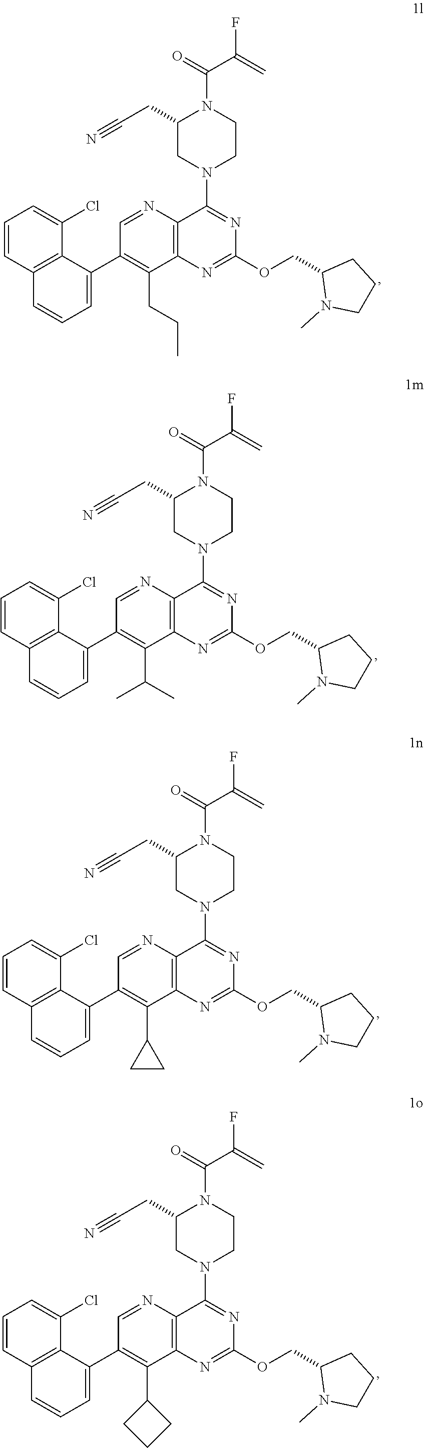

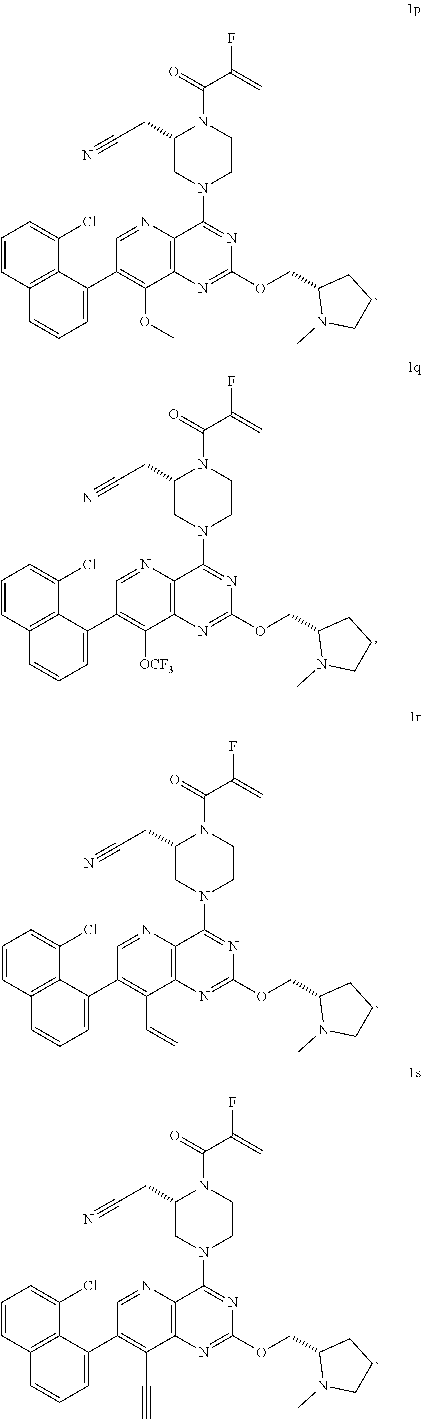

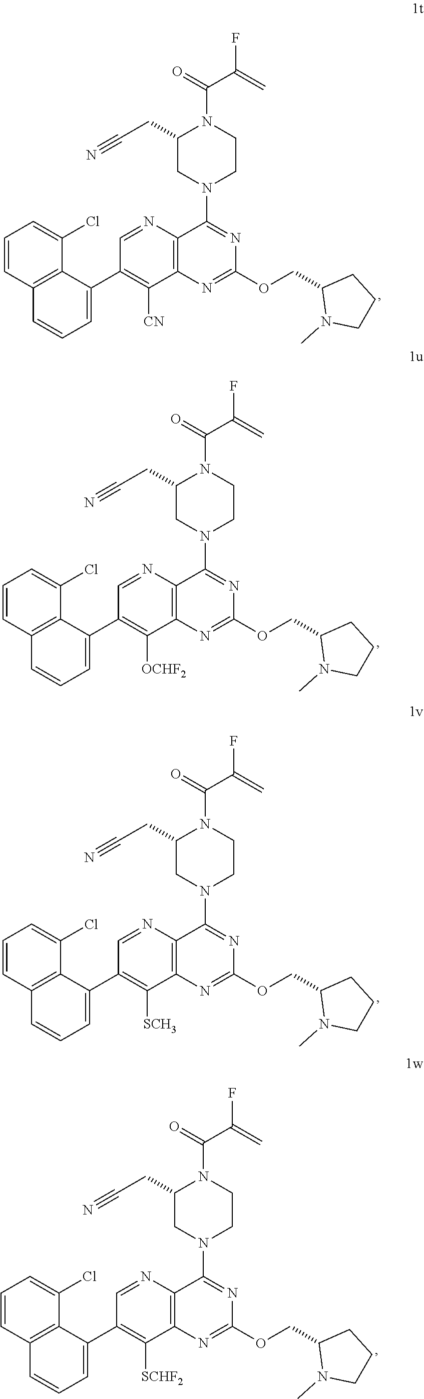

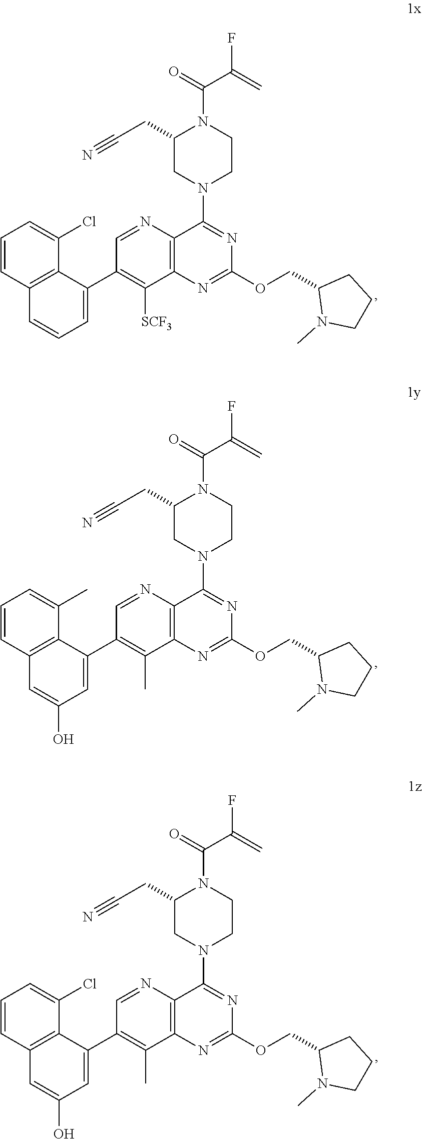

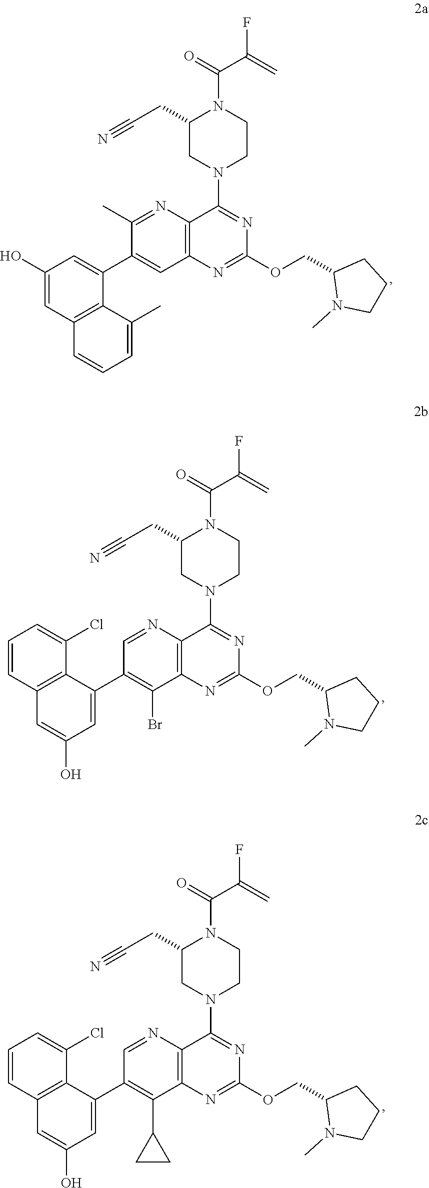

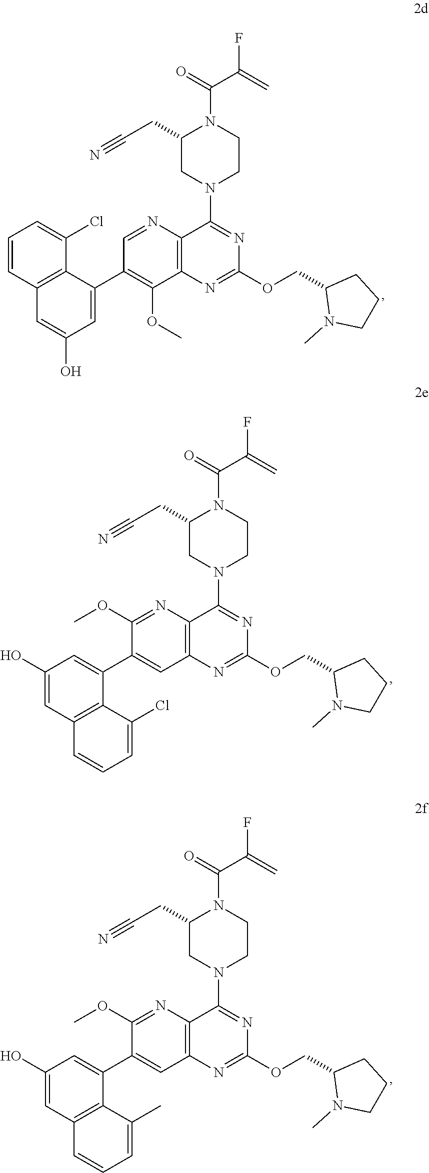

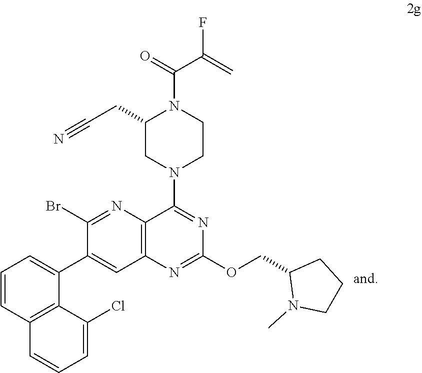

Particularly preferred compounds of the invention are compounds 1d, 1f, 1n and 1z:

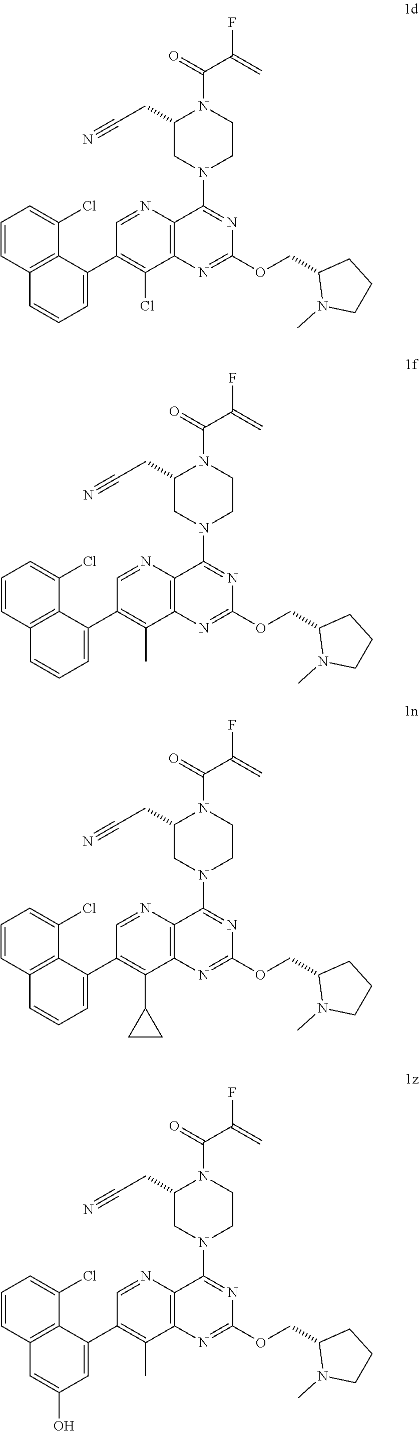

In one embodiment, the invention includes a pharmaceutical composition, comprising either Compound 1d, Compound 1f, Compound 1n or Compound 1z, or a salt, solvate, or prodrug thereof together with a pharmaceutically acceptable carrier.

Compounds disclosed herein can be administered as the neat chemical, but are preferably administered as a pharmaceutical composition. Accordingly, the invention encompasses pharmaceutical compositions comprising a compound or pharmaceutically acceptable salt of a compound, such as a compound of Formula I, together with at least one pharmaceutically acceptable carrier. The pharmaceutical composition may contain a compound or salt of Formula I as the only active agent, but is preferably contains at least one additional active agent. As will be appreciated by those skilled in the art, combinations of the various compounds described by Formula I may also be implemented in the compositions and methods of the invention. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formula I, and an additional active agent. For example, the pharmaceutical composition may contain a molar ratio of about 0.5:1, about 1:1, about 2:1, about 3:1 or from about 1.5:1 to about 4:1 of an additional active agent to a compound of Formula I.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt %) of a compound of Formula III and usually at least about 5 wt % of a compound of Formula I. Some embodiments contain from about 25 wt % to about 50 wt % or from about 5 wt % to about 75 wt % of the compound of Formula I.

### Treatment Methods

The compounds of Formula I, as well as pharmaceutical compositions comprising the compounds, are useful for diagnosis or treatment of a disease, disorder, or medical condition mediated through KRAS, especially the KRAS mutant G12C, and including various cancers, such as glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

According to the present invention, a method of KRAS-mediated diseases or conditions comprises providing to a patient in need of such treatment a therapeutically effective amount of a compound of Formula I. In one embodiment, the patient is a mammal, and more specifically a human. As will be understood by one skilled in the art, the invention also encompasses methods of treating non-human patients such as companion animals, e.g., cats, dogs, and livestock animals.

A therapeutically effective amount of a pharmaceutical composition is preferably an amount sufficient to reduce or ameliorate the symptoms of a disease or condition. In the case of KRAS-mediated diseases for example, a therapeutically effective amount may be an amount sufficient to reduce or ameliorate cancer. A therapeutically effective amount of a compound or pharmaceutical composition described herein will also provide a sufficient concentration of a compound of Formula I when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

According to the invention, the methods of treatment disclosed herein include providing certain dosage amounts of a compound or compounds of Formula I to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I are provided daily to a patient. Frequency of dosage may also vary depending on the compound used and the particular disease treated. However, for treatment of most KRAS-mediated diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I may be administered singularly (i.e., sole therapeutic agent of a regime) to treat or prevent KRAS-mediated diseases and conditions such as various cancers, or may be administered in combination with another active agent. One or more compounds of Formula I may be administered in coordination with a regime of one or more other active agents such as anticancer cytotoxic agents. In an embodiment, a method of treating or diagnosing KRAS-mediated cancer in a mammal includes administering to said mammal a therapeutically effective amount of a compound of Formula I, optionally in combination with one or more additional active ingredients.

As will be appreciated by one skilled in the art, the methods of treatment provided herein are also useful for treatment of mammals other than humans, including for veterinary applications such as to treat horses and livestock, e.g., cattle, sheep, cows, goats, swine and the like, and pets (companion animals) such as dogs and cats.

For diagnostic or research applications, a wide variety of mammals will be suitable subjects including rodents (e.g., mice, rats, hamsters), rabbits, primates, and swine such as inbred pigs and the like. Additionally, for in vitro applications, such as in vitro diagnostic and research applications, body fluids (e.g., blood, plasma, serum, cellular interstitial fluid, saliva, feces, and urine) and cell and tissue samples of the above subjects will be suitable for use.

In one embodiment, the invention provides a method of treating a disease, disorder, or medical condition mediated through KRAS, especially the KRAS mutant G12C. including various cancers, in a patient identified as in need of such treatment, the method comprising providing to the patient an effective amount of a compound of Formula I. The compounds of Formula I provided herein may be administered alone, or in combination with one or more other active agents.

In another embodiment, the method of treating or diagnosing KRAS-mediated diseases or conditions may additionally comprise administering the compound of Formula I in combination with one or more additional compounds, wherein at least one of the additional compounds is an active agent, to a patient in need of such treatment. The one or more additional compounds may include additional therapeutic compounds, including anticancer therapeutic compounds such as doxorubicin, paclitaxel, docetaxel, cisplatin, camptothecin, temozolomide, Avastin, Herceptin, Erbitux, and the like.

### EXAMPLES

### Chemical Synthesis

The synthesis of compounds of this invention is exemplified by the sequence of steps shown in Schemes 1-6. Scheme 1 illustrates the synthesis of examples of the Formula I where Y and G are hydrogen and X is O, NR², or S (i.e., 10a-10c). In Scheme 1, reaction of commercially available compound 3 with 4 in the presence of a base such as DIPEA in a solvent such as acetonitrile will produce 5. Reaction of 6a-6c with a base such as sodium hydride, Hünig's base, K₂CO₃ or a Cs₂CO₃/DABCO mixture followed by treatment with 5 in a polar aprotic solvent such as N-methyl-2-pyrrolidone at RT or elevated temperature generates compounds 7a-7c, respectively. A standard Suzuki coupling procedure between compounds 7a-7c and 8 in a solvent mixture such as 1,4-dioxane and water can be employed to prepare compounds 9a-9c. Removal of the Boc protecting group of 9a-9c under acidic conditions such as anhydrous HCl in 1,4-dioxane, followed by acylation of the deprotected product with an α,β-unsaturated acid chloride such as acryloyl chloride in a solvent such as methylene chloride containing a base such as triethylamine, will produce the corresponding compounds 10a-10c of the Formula I, where Y and G are hydrogen and X is either O, NR², or S.

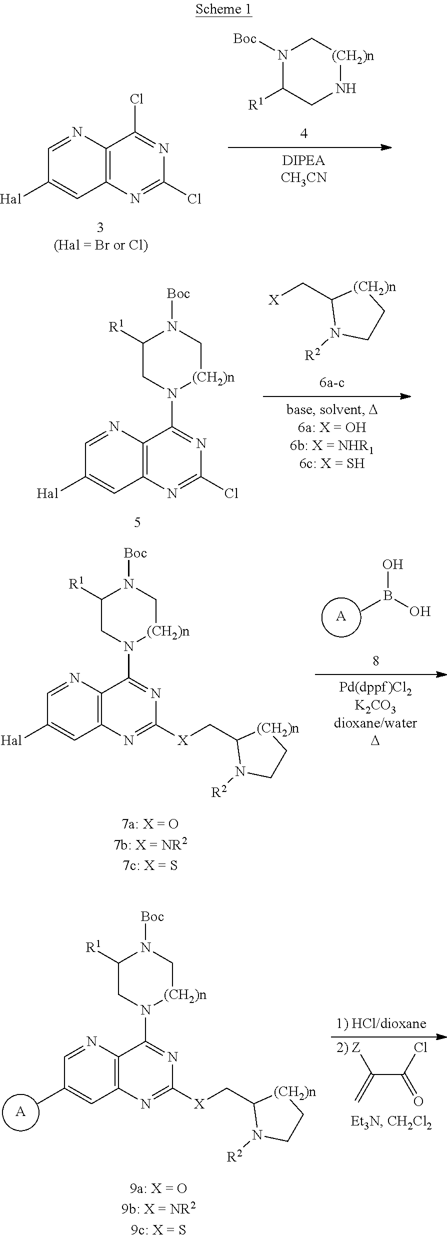

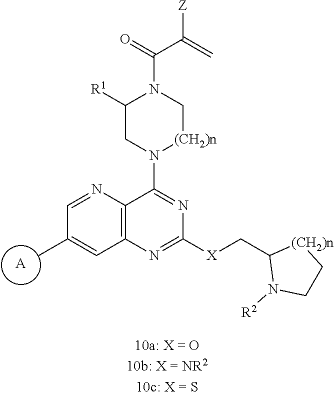

Scheme 2 illustrates the synthesis of examples of the Formula I where Y or G is fluorine and X is either O, NR², or S (i.e., 18a-c and 19a-c). Oxidation of commercially available 7-bromopyrido[3,2-d]pyrimidine-2,4-diol (11) with urea-hydrogen peroxide complex in the presence of trifluoroacetic anhydride at 0° C. in a aprotic solvent such as DMF provides N-oxide 12. Subsequent reaction of 12 with POCl₃ in the presence of Hünig's base generates a roughly 1:1 mixture of trichloro compounds 13a and 13b. Treatment of the 13a/13b mixture with 4 in the presence of Hünig's base in a solvent such as acetonitrile furnishes the corresponding products 14a and 14b, which may be separated by chromatography. Treatment of 6a-6c with a base such as sodium hydride, Hünig's base, K₂CO₃ or a Cs₂CO₃/DABCO mixture followed by reaction with 14a and 14b in a polar aprotic solvent such as N-methyl-2-pyrrolidone at RT or elevated temperature affords compounds 15a and 15b, respectively. Reaction of 15a and 15b with a fluoride source such as potassium fluoride or cesium fluoride at elevated temperature in a polar aprotic solvent like DMSO furnishes the corresponding fluoro products 16a and 16b. A standard Suzuki coupling procedure between compounds 16a and 16b and 8 in a solvent mixture such as 1,4-dioxane and water can be employed to prepare compounds 17a and 17b. Removal of the Boc protecting group of 17a and 17b under acidic conditions such as anhydrous HCl in 1,4-dioxane. Subsequent acylation of the deprotected product with an α,β-unsaturated acid chloride such as acryloyl chloride in a solvent such as methylene chloride containing a base such as triethylamine, will produce the corresponding compounds 18a-c and 19a-c of the Formula I where either Y or G is fluorine and X is either O, NR², or S.

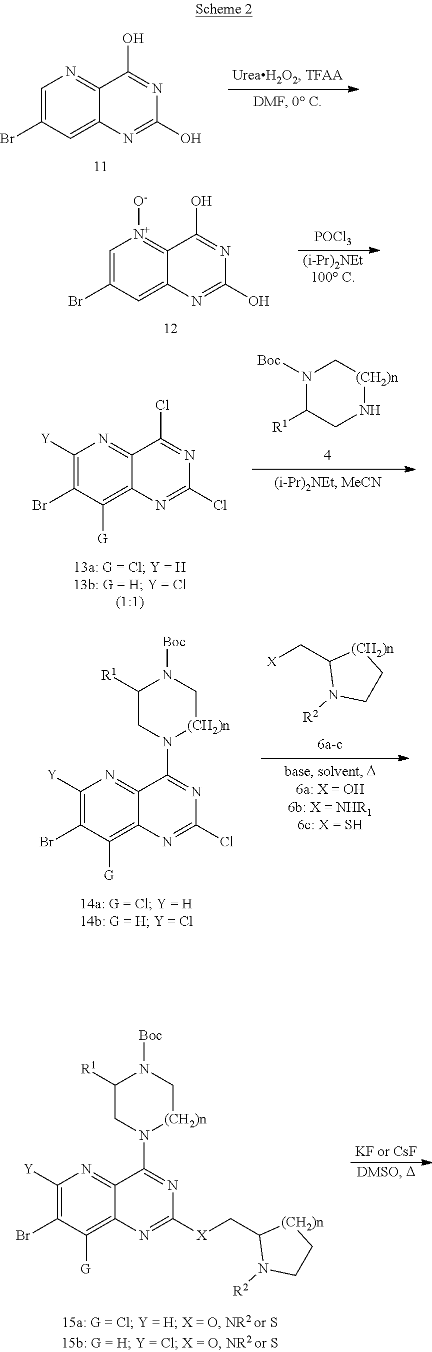

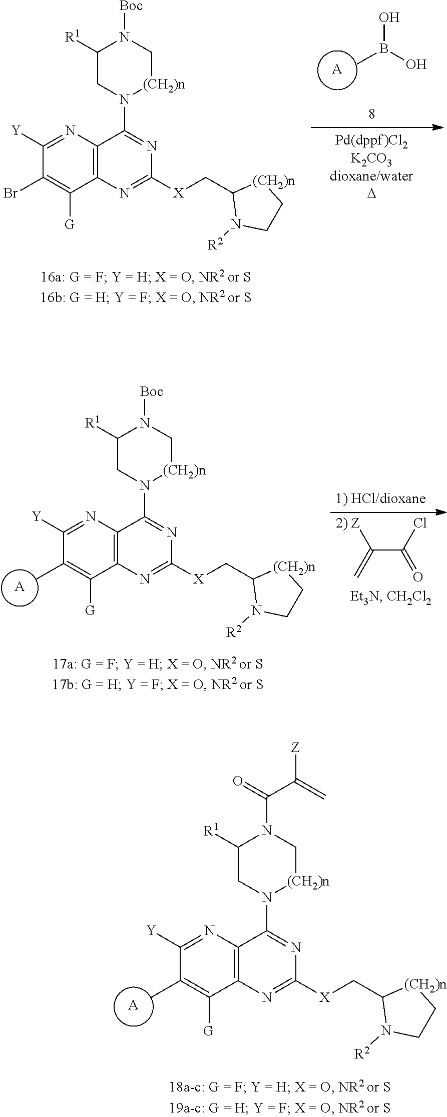

Scheme 3 illustrates the synthesis of examples of the Formula I where Y is hydrogen and G is fluorine and X is either O, NR², or S (i.e., 31a-31c). Reaction of either commercially available 3,5-dibromo-4-fluoropyridine or 3,5-dichloro-4-fluoropyridine (20) with (1Z)—N-[(methylsulfonyl)oxy]-ethanimidoyl chloride (21; CAS #1228558-17-5) according to the general procedure described by P. S. Fier (J. Am. Chem. Soc. 2017, 139 (28), 9499-9736) provides 3,5-dihalo-4-fluoropicolinonitrile (22). Alternatively, compound 22 can be prepared by the oxidation of 20 with H₂O₂-urea complex in the presence of trifluoroacetic anhydride followed by treatment of the corresponding N-oxides with trimethylsilyl cyanide in the presence of dimethylcarbamoyl chloride in a solvent such as dichloromethane. Regioselective Suzuki coupling of 22 with boronic acid 8 as generally described in WO2021117767A1 affords product 23. Subsequent reaction of 23 with 2,4-dimethoxybenzylamine (24) according to the procedure described in WO2021041671A1 in the presence of Hünig's base while heating in a suitable solvent such as 1,4-dioxane furnishes compound 25. Alternatively, 25 can be prepared by a Buchwald-Hartwig amination procedure between 23 and 24 under standard conditions. The Pinner reaction of 25 in methanol in the presence of HCl conducted at −78° C. to 0° C. followed by hydrolysis of the intermediate imino ester in the presence of saturated aqueous NaHCO₃ affords compound 26. Reaction of 26 with trichloroacetyl isocyanate at 0° C. followed by treatment with anhydrous ammonia in methanol and warming to room temperature provides compound 27. Reaction of 27 with POCl₃ in the presence of Hünig's base at elevated temperature yields the corresponding 2,4-dichloro-8-fluoropyrido[3,2-d]pyrimidine derivative 28. Reaction of compound 28 with 4 in the presence of Hünig's base in a solvent such as acetonitrile provides 29. Treatment of 6a-6c with a suitable base such as potassium fluoride, Hünig's base, K₂CO₃ or a Cs₂CO₃/DABCO mixture in either neat 6a-6c or in a suitable aprotic solvent followed by reaction with 29 at elevated temperature generates affords compounds 30a-30c, respectively. Alternatively, 6a and 29 can be coupled with Pd(OAc)₂ the presence of BINAP and Cs₂CO₃ in toluene at elevated temperature to produce 30a. Removal of the Boc protecting group of 30a-30c under acidic conditions such as anhydrous HCl in 1,4-dioxane. Acylation of the corresponding deprotected product with an α,β-unsaturated acid chloride such as acryloyl chloride in an aprotic solvent such as methylene chloride containing a base such as triethylamine, will generate compounds of the Formula I where Y is hydrogen, G is fluorine and X is O, NR², or S (i.e., 31a-31c).

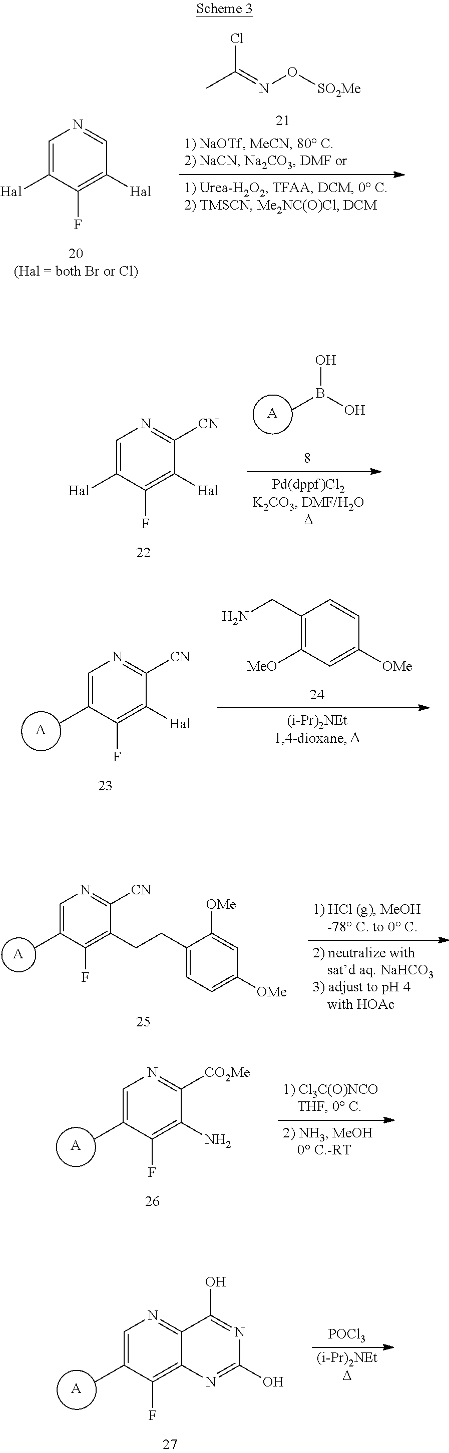

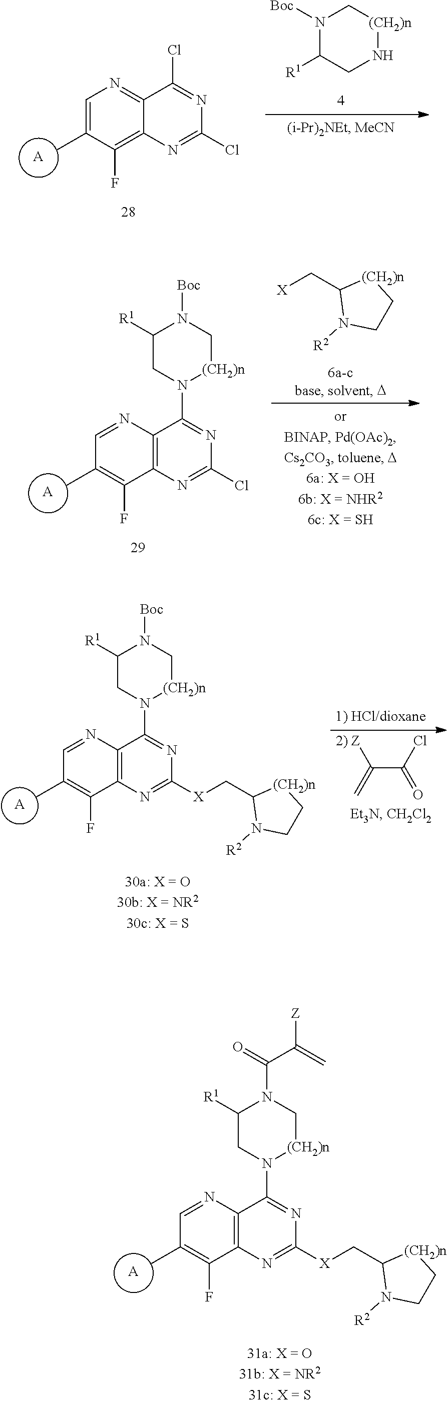

Scheme 4 illustrates the synthesis of examples of the Formula I where Y is hydrogen and G is hydrogen, halogen. C₁₋₄alkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl) and X is either O, NR², or S. Oxidation of 32 with urea-hydrogen peroxide complex in the presence of trifluoracetic anhydride follow by treatment with dimethylcarbamoyl chloride and trimethylsilyl cyanide provides nitrile 33. A standard Suzuki coupling procedure between 33 and 8 can be employed to prepare compound 34. Reaction of 34 with 2,4-dimethoxybenzylamine (35) at elevated temperature can furnish compound 36. The Pinner reaction of 36 in methanol in the presence of HCl conducted at −78° C. to 0° C. followed by hydrolysis of the intermediate imino ester in the presence of saturated aqueous NaHCO₃ affords ester 37. Reaction of 37 with trichloroacetyl isocyanate at 0° C. followed by treatment with anhydrous ammonia in methanol and warming to room temperature provides diol 38. Reaction of 38 with POCl₃ in the presence of Hünig's base at elevated temperature yields the dichloro compound 39. Reaction of compound 39 with 4 in the presence of Hünig's base in a solvent such as acetonitrile provides 40. Treatment of 6a-6c with a suitable base such as potassium fluoride, Hünig's base, K₂CO₃ or a Cs₂CO₃/DABCO mixture in either neat 6a-6c or in a suitable aprotic solvent followed by reaction with 40 at elevated temperature generates affords compounds 41a-41c, respectively. Alternatively, 6a and 40 can be coupled with Pd(OAc)₂ the presence of BINAP and Cs₂CO₃ in toluene at elevated temperature to produce 41a. Removal of the Boc protecting group of 41a-41c under acidic conditions such as anhydrous HCl in 1,4-dioxane. Acylation of the corresponding deprotected product with an α,β-unsaturated acid chloride such as acryloyl chloride in an aprotic solvent such as methylene chloride containing a base such as triethylamine, will generate compounds of the Formula I where Y is hydrogen and G is hydrogen, halogen, C₁₋₄alkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl) and X is either O, NR², or S (i.e., 42a-42c).

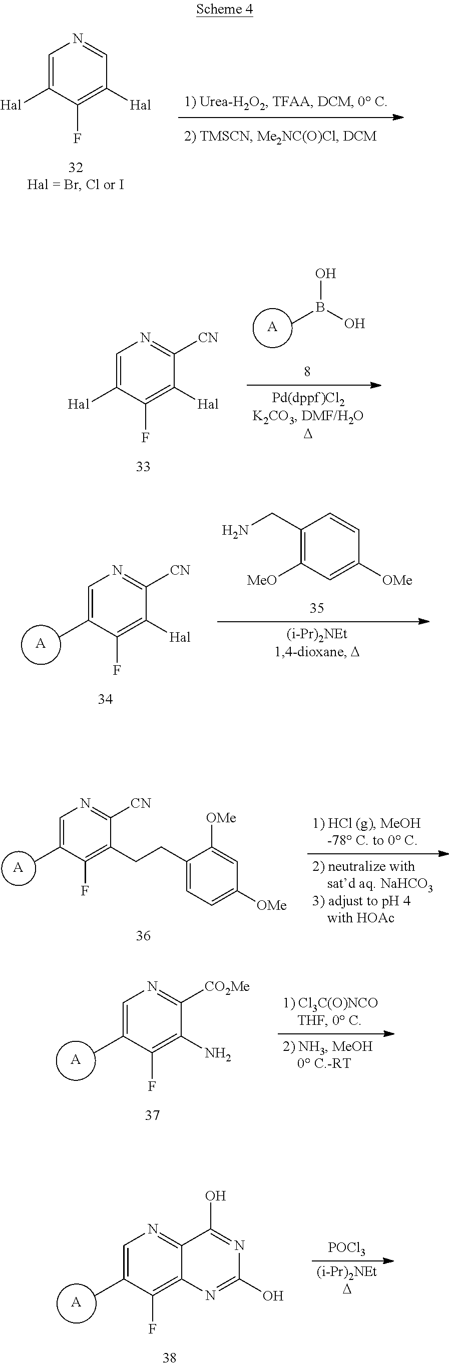

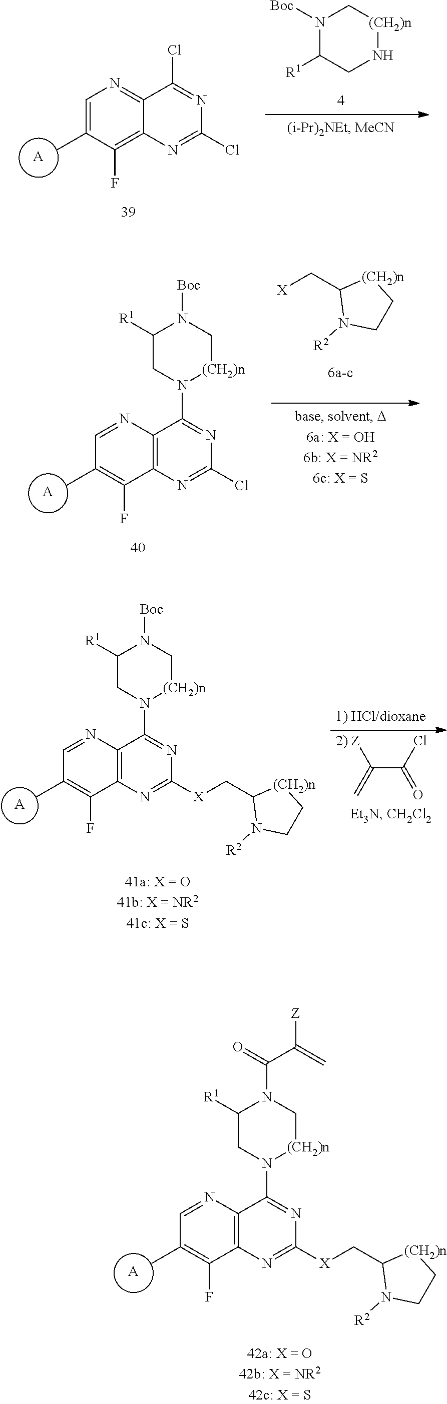

Scheme 5 illustrates the synthesis of examples of the Formula I where Y and G are hydrogen, halogen. C₁₋₄alkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl) and X is methylene (i.e., 46). The reaction of acetylene 44 with a strong base such as sodium hydride generates the corresponding acetylide anion, which can then be reacted with 43 to provide 45. Alternatively, Sonogashira coupling of 43 with 44 using a Pd catalyst such as Pd(dppf)₂Cl₂ can furnish compound 45. Catalytic hydrogenation of 45 followed by removal of the Boc group under acidic conditions such as TFA in dichloromethane affords the corresponding amine, which can be subsequently reacted with an acryloyl chloride in a solvent such as methylene chloride containing a base such as triethylamine to generate compounds of the Formula I where Y and G are either hydrogen, halogen, C₁₋₄alkyl, C₀-C₂alkenyl, C₀-C₂alkynyl, —(C₀-C₂alkyl)cycloalkyl, C₁₋₄haloalkyl, —O(C₁₋₄alkyl), —S(C₁₋₄alkyl), C₀-C₂ cyano, or —O(C₁₋₄ haloalkyl) and X is methylene (i.e., 46).

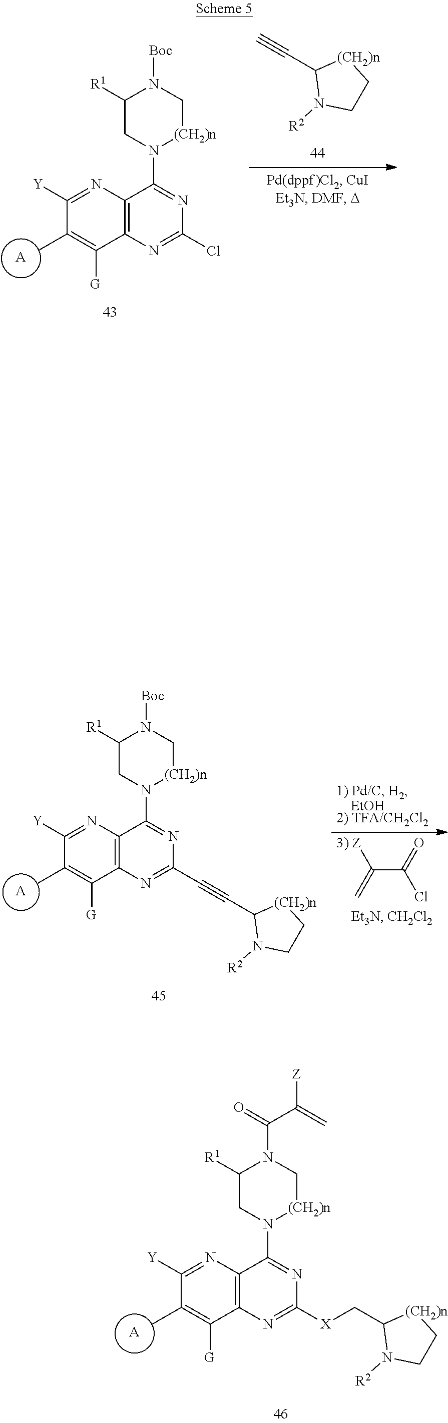

Scheme 6 illustrates the synthesis of examples of the Formula I where Y is hydrogen, halogen, C₁₋₄alkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl), G is hydrogen and X is either O, NR², or S. Nitration of picolinic acid 47 with nitric acid in concentrated sulfuric acid provides the corresponding nitro compound 48. Esterification of 48 with catalytic sulfuric acid in methanol at reflux generates methyl ester 49. Reduction of the nitro group of 49 with stannous chloride dihydrate in the presence of hydrochloric acid in an alcohol such as ethanol furnishes the corresponding amino derivative 50. Hydrolysis of the ester moiety of 50 in a wet solvent such as acetonitrile or THF containing 6% water (v/v) in the presence of LiBr and triethylamine provides carboxylic acid 51 (S. Karlsson et al, Tet. Lett. 2007 48, 2497-2499). Treatment of 51 with HATU and ammonium chloride in the presence of sodium bicarbonate in a polar aprotic solvent such as DMF generates the corresponding carboxamide derivative 52. Reaction of 52 with triphosgene (bis(trichloromethyl) carbonate in an aprotic solvent such as 1,4-dioxane at 5° C. followed by heating at 110° C. yields 7-bromopyrido[3,2-d]pyrimidine-2,4 (1H,3H)-dione derivative 53. Reaction of 53 with phosphorous oxychloride in the presence of Hünig's base at 120° C. provides the corresponding dichloro derivative 54. Reaction of 54 with 4 in the presence of Hünig's base in a solvent such as acetonitrile provides 55. Treatment of 6a-6c with a suitable base such as sodium hydride, Hünig's base, K₂CO₃ or a Cs₂CO₃/DABCO mixture followed by reaction with 55 at elevated temperature generates affords compounds 56a-56c, respectively. Alternatively, 6a and 55 can be coupled with Pd(OAc)₂ the presence of BINAP and Cs₂CO₃ in toluene at elevated temperature to produce 56a. A standard Suzuki coupling procedure between 56a-56c and 8 can be employed to prepare compounds 57a-57c. Removal of the Boc protecting group of 57a-57c under acidic conditions such as anhydrous HCl in 1,4-dioxane followed by acylation of the corresponding deprotected product with an α,β-unsaturated acid chloride such as acryloyl chloride in an aprotic solvent such as methylene chloride containing a base such as triethylamine, will generate compounds of the Formula I where Y is hydrogen, halogen, C₁₋₄alkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl), G is hydrogen and X is either O, NR², or S (i.e., 58a-58c).

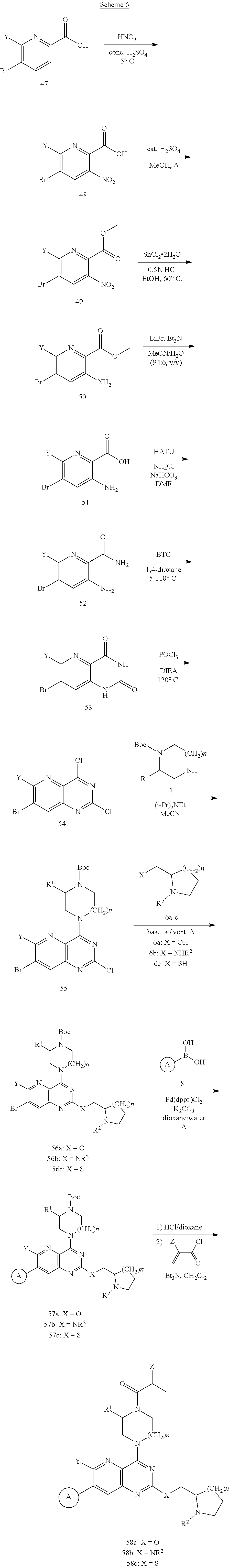

### Abbreviations and Acronyms

The following abbreviations and acronyms may be used in this application:
ACN=acetonitrile;
anhyd.=anhydrous;
aq.=aqueous;
B2pin2=bis(pinacolato)diboron;
Boc=tert-butoxycarbonyl;
BTC=triphosgene (bis(trichloromethyl) carbonate
n-Bu3P=tri-n-butylphosphine;
Compd=compound;
d=day(s);
DABCO=1,4-diazabicyclo[2.2.2]octane;
DCM=dichloromethane;
DIEA=DIPEA=N,N-diisopropylethylamine=Hünig's base;
DMF=N,N-dimethylformamide;
DMSO=dimethylsulfoxide;
DMA=N,N-dimethylacetamide;
dppf=1,1′-bis(diphenylphosphino)ferrocene)
EtOAc=ethyl acetate;
equiv=equivalents;
Ex=Example;
h=hour(s);
HATU=1-[bis(dimethylamino)methylene]-1H-1,2,3-triazolo[4,5-b]pyridinium 3-oxide hexafluorophosphate
LiHMDS=lithium bis(trimethylsilyl)amide [LiN(SiMe₃)₂];
MeOH=methanol;
NMP=N-methyl-2-pyrrolidone;
min=minutes;
Pd(dppf)Cl₂=[1,1′-bis(diphenylphosphino)ferrocene]dichloropalladium (II);
PE=petroleum ether;
RT=room temperature;
satd.=saturated solution;
TEA=triethylamine;
TFA=trifluoroacetic acid;
TFAA=trifluoroacetic anhydride;
THF=tetrahydrofuran;
TMSCN=trimethylsilyl cyanide.

### EXAMPLES

The present inventive concept has been described in terms of exemplary principles and embodiments, but those skilled in the art will recognize that variations may be made and equivalents substituted for what is described without departing from the scope and spirit of the disclosure as defined by the following examples.

### Example 1

### 2-((S)-4-(7-(8-Chloronaphthalen-1-yl)-2-(((S)-1-methylpyrrolidin-2-yl)methoxy)pyrido[3,2-d]pyrimidin-4-yl)-1-(2-fluoroacryloyl)piperazin-2-yl)acetonitrile (1a)

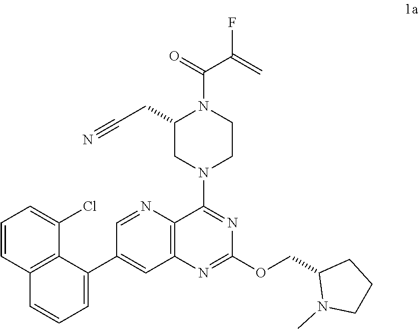

Example 1 (1a) was prepared as shown below in Scheme 7.

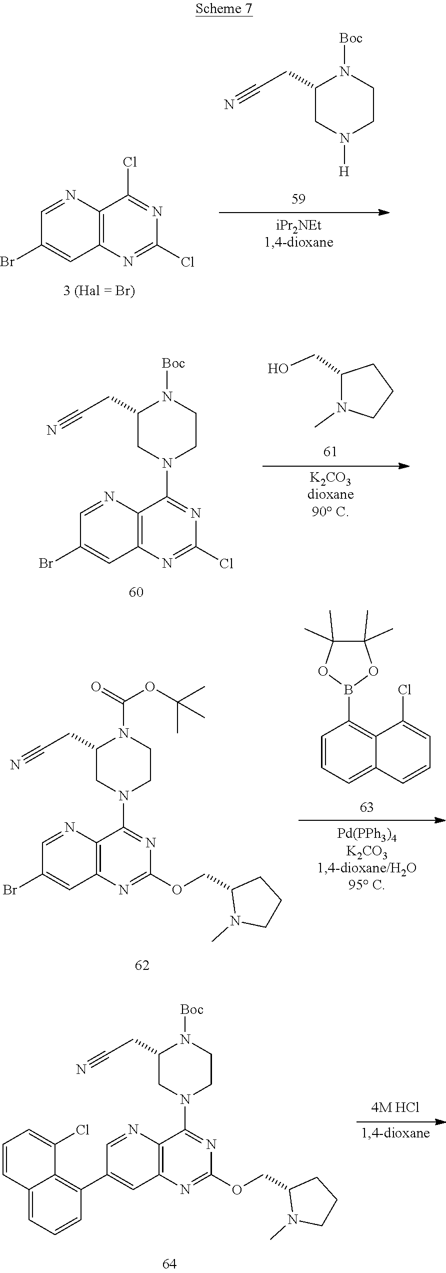

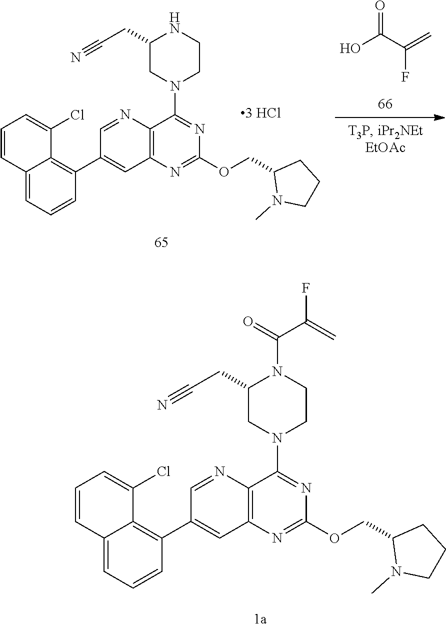

### tert-Butyl(S)-4-(7-bromo-2-chloropyrido[3,2-d]pyrimidin-4-yl)-2-(cyanomethyl)piperazine-1-carboxylate (60)

A mixture of 7-bromo-2,4-dichloropyrido[3,2-d]pyrimidine (3 where Hal=Br; CAS #1215074-41-1; 1.00 g, 3.61 mmol) and tert-butyl (2S)-2-(cyanomethyl)piperazine-1-carboxylate (59; CAS #1589565-36-5; 0.90 g, 3.97 mmol) in anhydrous 1,4-dioxane (9.0 mL) was treated with diisopropylethylamine (1.90 mL, 10.8 mmol) slowly dropwise at RT while stirring. After 1 h, the reaction mixture was concentrated in vacuo and the crude product was purified by silica gel column chromatography eluting with a gradient of 5-50% EtOAc in hexane to afford 1.64 g (98%) of tert-butyl(S)-4-(7-bromo-2-chloropyrido[3,2-d]pyrimidin-4-yl)-2-(cyanomethyl)piperazine-1-carboxylate (60) as a light-yellow solid: HPLC-MS (ES⁺) m/z MH⁺=467.

### tert-Butyl(S)-4-(7-bromo-2-(((S)-1-methylpyrrolidin-2-yl)methoxy)pyrido[3,2-d]pyrimidin-4-yl)-2-(cyanomethyl)piperazine-1-carboxylate (62)

A mixture of tert-butyl(S)-4-(7-bromo-2-chloropyrido[3,2-d]pyrimidin-4-yl)-2-(cyanomethyl)piperazine-1-carboxylate (60; 500 mg, 1.07 mmol) and(S)-(1-methylpyrrolidin-2-yl) methanol (61; CAS #34381-71-0; 0.20 mL, 1.60 mmol) in anhydrous 1,4-dioxane (6.0 mL) was treated with K₂CO₃ (443 mg. 3.21 mmol) and the reaction mixture heated at 90° C. with stirring under a N₂ atmosphere for 16 h. The reaction mixture was cooled to RT, concentrated in vacuo and the crude product was purified by silica gel column chromatography eluting with a gradient of 0-10% MeOH in DCM to afford 370 mg (63%) of tert-butyl(S)-4-(7-bromo-2-(((S)-1-methylpyrrolidin-2-yl)methoxy)pyrido[3,2-d]pyrimidin-4-yl)-2-(cyanomethyl)piperazine-1-carboxylate (62) as an off-white solid: HPLC-MS (ES⁺) m/z MH⁺=546.

### tert-Butyl(S)-4-(7-(8-chloronaphthalen-1-yl)-2-(((S)-1-methylpyrrolidin-2-yl)methoxy)pyrido[3,2-d]pyrimidin-4-yl)-2-(cyanomethyl)piperazine-1-carboxylate (64)

A mixture of tert-butyl(S)-4-(7-bromo-2-(((S)-1-methylpyrrolidin-2-yl)methoxy)pyrido[3,2-d]pyrimidin-4-yl)-2-(cyanomethyl)piperazine-1-carboxylate (62; 450 mg. 0.825 mmol), 2-(8-chloronaphthalen-1-yl)-4,4,5,5-tetramethyl-1,3,2-dioxaborolane (63; 950 mg. 3.30 mmol), and K₂CO₃ (494 mg. 3.30 mmol) in 1,4-dioxane (7.0 mL) and water (0.9 mL) was degassed by sparging with N₂ with stirring for 20 minutes. Tetrakis(triphenylphosphine) palladium (0) (143 mg. 0.123 mmol) was added and the reaction mixture degassed by sparging again with N₂ with stirring for an additional 15 minutes. The reaction mixture was heated at 80° C. with stirring under a N₂ atmosphere for 16 h, cooled to RT, diluted with EtOAc and then filtered through Celite. The filtrate was washed with satd. aq. NaCl (3×), dried (MgSO₄), filtered and concentrated in vacuo. The crude product was purified by silica gel column chromatography eluting with a gradient of 0-15% MeOH in DCM to afford 130 mg (25%) of tert-butyl(S)-4-(7-(8-chloronaphthalen-1-yl)-2-(((S)-1-methylpyrrolidin-2-yl)methoxy)pyrido[3,2-d]pyrimidin-4-yl)-2-(cyanomethyl)piperazine-1-carboxylate (64) as a beige solid: HPLC-MS (ES⁺) m/z MH⁺=628; ¹H NMR (300 MHz, CDCl₃) δ 8.54 (dd, J=3.2, 1.2 Hz, 1H), 7.97 (dd, J=8.2, 1.1 Hz, 1H), 7.91 (d, J=2.2 Hz, 1H), 7.89 (dd, J=7.0, 1.2 Hz, 1H), 7.60-7.53 (m, 2H), 7.47-7.40 (m, 2H), 5.97 (br s, 1H), 5.35 (br s, 1H), 4.69 (br s. 1H), 4.55-4.47 (m, 1H), 4.35-4.25 (m, 1H), 4.15 (br s, 1H), 3.62 (br s, 1H), 3.26 (br s, 2H), 3.14-3.08 (m, 1H), 2.94-2.86 (m, 1H), 2.79-2.61 (m, 2H), 2.50 (s, 3H), 2.35-2.24 (m, 1H), 2.15-2.00 (m, 1H), 1.92-1.76 (m, 3H), 1.52 (s, 9H).

### 2-((S)-4-(7-(8-Chloronaphthalen-1-yl)-2-(((S)-1-methylpyrrolidin-2-yl)methoxy)pyrido[3,2-d]pyrimidin-4-yl)piperazin-2-yl)acetonitrile hydrochloride (1:3) (65)

A mixture of tert-butyl(S)-4-(7-(8-chloronaphthalen-1-yl)-2-(((S)-1-methylpyrrolidin-2-yl)methoxy)pyrido[3,2-d]pyrimidin-4-yl)-2-(cyanomethyl)piperazine-1-carboxylate (64; 160 mg, 0.255 mmol) and 4 M HCl in 1,4-dioxane (2.0 mL) was stirred at RT under a N₂ atmosphere. After 1 h, the reaction mixture was concentrated in vacuo to afford 180 mg (93%) of 2-((S)-4-(7-(8-chloronaphthalen-1-yl)-2-(((S)-1-methylpyrrolidin-2-yl)methoxy)pyrido[3,2-d]pyrimidin-4-yl)piperazin-2-yl)acetonitrile hydrochloride (1:3) (65) as a beige solid: HPLC-MS (ES⁺) m/z MH⁺=528; ¹H NMR (300 MHz, DMSO-d₆) δ 11.1 (br s, 1H), 10.3 (br s, 2H), 8.68 (d, J=1.6 Hz, 1H), 8.22 (dd, J=7.2, 1.1 Hz, 1H), 8.14 (dd, J=8.1, 1.2 Hz, 1H), 8.08 (d, J=2.1 Hz, 1H), 7.77-7.67 (m, 2H), 7.65-7.53 (m, 2H), 4.91-4.74 (m, 2H), 4.23 (br s, 2H), 3.92-3.83 (m, 2H), 3.74-3.66 (m, 1H), 3.51-3.43 (m, 2H), 3.28 (br s, 4H), 3.18-3.05 (m, 1H), 2.96 (d, J=4.7 Hz, 3H), 2.36-2.22 (m, 1H), 2.14-1.77 (m, 4H).

### 2-((S)-4-(7-(8-Chloronaphthalen-1-yl)-2-(((S)-1-methylpyrrolidin-2-yl)methoxy)pyrido[3,2-d]pyrimidin-4-yl)-1-(2-fluoroacryloyl)piperazin-2-yl)acetonitrile (1a)

A mixture of 2-((S)-4-(7-(8-chloronaphthalen-1-yl)-2-(((S)-1-methylpyrrolidin-2-yl)methoxy)pyrido[3,2-d]pyrimidin-4-yl)piperazin-2-yl)acetonitrile hydrochloride (1:3) (65; 100 mg, 0.157 mmol), 2-fluoroprop-2-enoic acid (66; CAS #430-99-9; 28 mg, 0.314 mmol), and oven-dried 4 Å molecular sieves (132 mg) in EtOAc (2.0 mL) was treated with diisopropylethylamine (0.22 mL, 1.26 mmol) and the reaction mixture stirred at RT. After 5 minutes, 1-propanephosphonic anhydride solution (T₃P, 0.33 mL, 0.471 mmol, 50% in EtOAc) was added and the reaction mixture stirred at RT. After 20 minutes, the reaction mixture was diluted with EtOAc, washed with 5% aq. NaHCO₃ (3×), dried (MgSO₄), filtered, and concentrated in vacuo. The crude product was purified by silica gel column chromatography eluting with a gradient of 10-100% EtOAc containing 1% Et₃N (v/v) in DCM to afford 40 mg (35%) of 2-((S)-4-(7-(8-chloronaphthalen-1-yl)-2-(((S)-1-methylpyrrolidin-2-yl)methoxy)pyrido[3,2-d]pyrimidin-4-yl)-1-(2-fluoroacryloyl)piperazin-2-yl)acetonitrile (1a) as an off-white solid: HPLC-MS (ES⁺) m/z MH⁺=600; ¹H NMR (300 MHz, CDCl₃) δ 8.57 (dd, J=2.1, 1.3 Hz, 1H), 7.98 (dd, J=8.2, 1.2 Hz, 1H), 7.93 (d, J=2.1 Hz, 1H), 7.90 (dd, J=8.1, 1.2 Hz, 1H), 7.60-7.54 (m, 2H), 7.48-7.40 (m, 2H), 5.44 (d, J=47.8 Hz, 1H), 5.26 (dd, J=13.3, 3.6 Hz, 1H), 4.65×A), 2.56 (s, 3H), 2.44-2.30 (m, 1H), 2.18-2.03 (m, 1H), 1.95-1.52 (m, 10H).

Intermediate 71 prepared as depicted in Scheme 8 is useful for preparing compounds having an 8-methyl substituent.

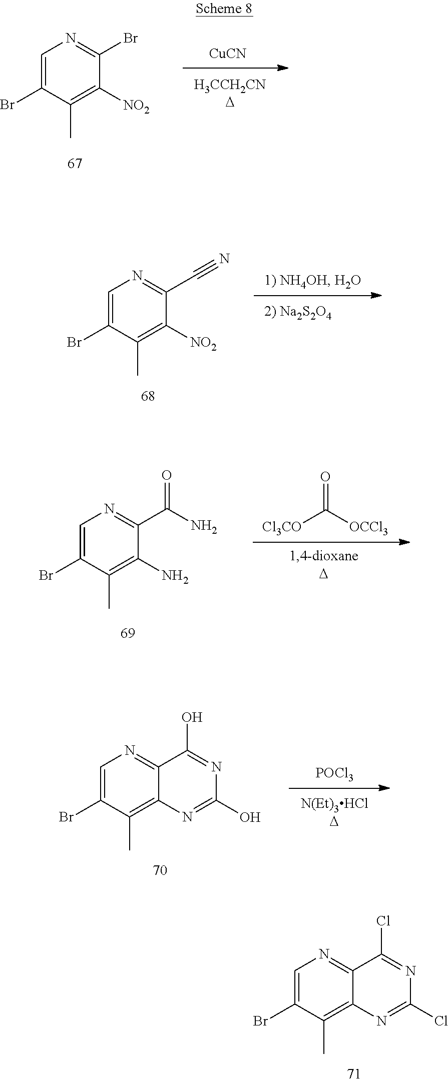

### 5-Bromo-3-nitro-4-methylpicolinonitrile (68)

Cuprous cyanide (1.83 g, 20.4 mmol) was added at RT to a stirred solution of 2,5-dibromo-3-nitro-4-methylpyridine (67; 5.04 g, 17.0 mmol) in propionitrile (34 mL). The resulting suspension was heated while stirring rapidly at reflux under nitrogen for 4 days. The cooled reaction mixture was extracted with a mixture of water and ethyl acetate and then filtered through celite. The phases of the filtrate were then separated and the ethyl acetate extract was washed with saturated NaCl (aq.), dried (CaSO₄), and evaporated to provide 3.9 g of crude product as a dark brown oil. This was purified by chromatography on silica gel eluting with a gradient of 1 to 5% EtOAc in hexanes to furnish 3.1 g of a yellow solid. This material was recrystallized from heptane to yield 2.7 g of 5-bromo-3-nitro-4-methylpicolinonitrile (68) as a white powder. HPLC-MS (ES⁻) m/z [M−H]⁻=240, 242; ¹H NMR (300 MHz, DMSO) δ 9.18 (s, 1H), 2.52 (s, 3H); ¹³C NMR (75 MHz, DMSO) δ 154.9, 149.7, 143.5, 129.8, 125.9, 114.0, 19.4.

### 3-Amino-5-bromo-4-methylpicolinamide (69)

Concentrated ammonium hydroxide (5.0 mL) was added to a stirred suspension of 5-bromo-3-nitro-4-methylpicolinonitrile (68; 1.20 g, 5.0 mmol) in water (10 mL). The flask was sealed with a septum cap and the suspension was rapidly stirred at RT. After 3 h, the suspension was treated over 15 minutes with portionwise addition of powdered 85% sodium hydrosulfite (4.8 g, 27.3 mmol) and stirred at RT for 2.5 h. The resulting yellow solid was isolated by filtration, washed with water and dried to provide 511 mg of crude product that was contaminated with ca. 25% 6-bromo-7-methyl-1,2-dihydro-3H-pyrazolo[4,3-b]pyridin-3-one. This material was purified by chromatography on silica gel eluting with a gradient of 0 to 5% MeOH in DCM to yield 328 mg of 3-amino-5-bromo-4-methylpicolinamide (69) as a white solid: HPLC-MS (ES⁺) m/z MH⁺=230, 232; ¹H NMR (300 MHz, DMSO) δ 7.99 (s, 1H), 7.89 (s, 1H), 7.45 (s, 1H), 7.15 (bs, 2H), 2.25 (s, 3H): ¹³C NMR (75 MHZ, DMSO) δ 170.5, 146.7, 136.6, 132.1, 127.7, 126.1, 17.0.

### 7-Bromo-8-methylpyrido[3,2-d]pyrimidine-2,4-diol (70)

(270 mg, 0.9 mmol) was added to a stirred solution of 3-amino-5-bromo-4-methylpicolinamide (69; 209 mg, 0.9 mmol) in anhydrous 1,4-dioxane (18 mL) and heated at reflux under nitrogen for 2 h. The cooled reaction mixture was partitioned between concentrated ammonium hydroxide and ethyl acetate. The resulting insoluble white precipitate was isolated by filtration, washed with water and air-dried on the filter paper to afford 151 mg of 7-bromo-8-methylpyrido[3,2-d]pyrimidine-2,4-diol (70). HPLC-MS (ES⁺) m/z MH⁺=256, 258; ¹H NMR (300 MHZ, DMSO) δ 8.39 (s, 1H), 7.74 (bs, 2H), 2.43 (s, 3H). ¹³C NMR (75 MHZ, DMSO) δ 162.1, 152.2, 144.2, 142.1, 135.2, 131.0, 127.4, 17.3.

### 7-Bromo-2,4-dichloro-8-methylpyrido[3,2-d]pyrimidine (71)

Phosphoryl chloride (22.5 mL, 241.4 mmol) was added to a stirred mixture of 7-bromo-8-methylpyrido[3,2-d]pyrimidine-2,4-diol (70; 1.24 g, 4.8 mmol) and triethylamine hydrochloride (2.7 g, 19.3 mmol) contained in a 50 mL round bottomed flask and heated at reflux for 4 h under. The resulting black solution was cooled and cautiously quenched by the slow addition to water at 0° C. Once the addition was complete, the product was extracted with ethyl acetate. The ethyl acetate extract was washed with saturated NaCl (aq.), dried (CaSO₄) and concentrated in vacuo to afford 2.8 g of 7-bromo-2,4-dichloro-8-methylpyrido[3,2-d]pyrimidine (71) as a light brown solid which was used without further purification: HPLC-MS (ES⁺) m/z MH⁺=294.

Intermediate 77 prepared as depicted in Scheme 9 is useful for preparing compounds having an 8-chloro substituent.

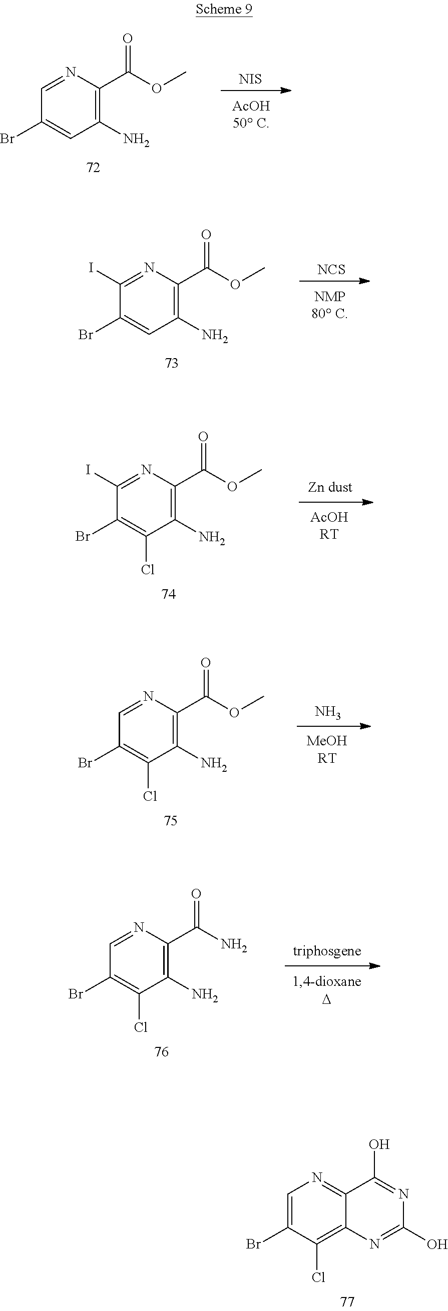

### Methyl 3-amino-5-bromo-6-iodopicolinate (73)

N-Iodosuccinimide (5.88 g, 26.0 mmol) was added to a stirred suspension of methyl 3-amino-5-bromopicolinate (72; CAS #1072448-08-8; 5.0 g, 21.7 mmol) in acetic acid (12.6 mL) and the mixture stirred at 50° C. After 5 d, the mixture was cooled to RT and slowly pipetted into satd. aq. NaHCO₃ and stirred at RT. After 2 h, the precipitated solid was filtered and washed with EtOAc to afford 2 g of methyl 3-amino-5-bromo-6-iodopicolinate (73) as a beige solid. The aqueous layer was extracted with EtOAc (3×), washed with satd. aq. NaHCO₃ (2×), 2% aq. Na₂S₂O₅ (3×), dried (MgSO₄), filtered and concentrated. Additional solid had precipitated from the filtrate after standing overnight. The solid was recrystallized from EtOAc to afford 1.0 g of methyl 3-amino-5-bromo-6-iodopicolinate (73) as a beige solid. The product from the extract was purified silica gel column chromatography eluting with a gradient of 10% to 40% EtOAc in hexanes to afford 1.13 g of methyl 3-amino-5-bromo-6-iodopicolinate (73) as a beige solid: HPLC-MS (ES⁺) m/z [M+H⁺]=357, 359; ¹H NMR (300 MHz, CDCl₃) δ 7.28 (s, 1H), 5.81 (br s, 2H), 3.94 (s, 3H).

### Methyl 3-amino-5-bromo-4-chloro-6-iodopicolinate (74)

N-Chlorosuccinimide (2.76 g, 20.6 mmol) was added to a stirred solution of methyl 3-amino-5-bromo-6-iodopicolinate (73; 1.53 g, 20.6 mmol) in N-methyl-2-pyrrolidone (20 mL). The mixture was placed into a metallic heating mantle that was preheated to 80° C. and stirred at 80° C. After 30 minutes, the red-orange mixture was quickly removed from the heating source and diluted with EtOAc. The mixture was washed satd. aq. NaCl (5×), dried (MgSO₄), filtered and concentrated in vacuo. The crude product was purified by silica gel column chromatography eluting with dichloromethane to afford 460 mg of methyl 3-amino-5-bromo-4-chloro-6-iodopicolinate (74) as a fluffy white solid: HPLC-MS (ES⁺) m/z [M+H⁺]=391, 393, 395; ¹H NMR (300 MHZ, CDCl₃) δ 6.45 (br s, 2H), 3.97 (s, 3H).

### Methyl 3-amino-5-bromo-4-chloropicolinate (75)

Zinc dust (51 mg, 0.79 mmol) was added to a stirred suspension of methyl 3-amino-5-bromo-4-chloro-6-iodopicolinate (74; 102 mg, 0.26 mmol) in acetic acid (2.4 mL) and the resulting dark green mixture stirred at RT. After 4 d the mixture had formed a tan suspension and an additional 14.4 mg of zinc dust was added. The mixture stirred at RT for 16 h and then poured into satd. aq. NaHCO₃, extracted with EtOAc (3×), dried (MgSO₄) filtered, and concentrated in vacuo. The crude product was purified by silica gel column chromatography eluting with a gradient of 0% to 10% EtOAc in DCM to afford 30 mg of methyl 3-amino-5-bromo-4-chloropicolinate (75) as an off-white solid: HPLC-MS (ES⁺) m/z [M+H⁺]=265, 267, 269; ¹H NMR (300 MHZ, CDCl₃) δ 8.16 (s, 1H), 6.45 (br s, 2H), 3.99 (s, 3H).

### 3-Amino-5-bromo-4-chloropicolinamide (76)

A mixture of methyl 3-amino-5-bromo-4-chloropicolinate (75; 200 mg, 0.753 mmol) and a solution of anhydrous ammonia in MeOH (4.0 mL, 7 N) was stirred at RT in a sealed flask. After 16 h, a white suspension had formed and the reaction mixture was concentrated in vacuo to afford 188 mg of 3-amino-5-bromo-4-chloropicolinamide (76) as a white solid: HPLC-MS (ES⁺) m/z [M+H⁺]=250, 252, 254; ¹H NMR (300 MHZ, DMSO-d₆) δ 8.09 (br s, 1H), 8.01 (s, 1H), 7.63 (br s, 1H), 7.41 (br s, 2H).

### 7-Bromo-8-chloropyrido[3,2-d]pyrimidine-2,4-diol (77)

Triphosgene (223 mg, 0.751 mmol) was added to a stirred solution of 3-amino-5-bromo-4-chloropicolinamide (76; 188 mg. 0.751 mmol) in anhydrous 1,4-dioxane (7.0 mL) and the mixture heated at reflux under N₂. After 5 h, additional triphosgene (227 mg) was added and the mixture heated at reflux for 16 h, cooled to RT and then concentrated in vacuo. The crude solid was triturated with EtOAc, filtered, and washed with EtOAc to afford 166 mg of 7-bromo-8-chloropyrido[3,2-d]pyrimidine-2,4-diol (77) as a gray solid: HPLC-MS (ES⁺) m/z [M+H⁺]=276, 278, 280; [M−H⁺]=274, 276, 278; ¹H NMR (300 MHz, DMSO-d₆) δ 11.8 (br s, 1H), 11.1 (br s, 1H), 8.66 (s, 1H).

### Nucleotide Exchange Assay

The biological activity of the Examples was determined in a KRAS G12C/SOS1 Nucleotide Exchange Assay that was performed by Reaction Biology Corporation (RBC), 1 Great Valley Parkway, Suite 2 Malvern, PA 19355, USA. The assay evaluates the SOS1-mediated Bodipy-GDP to GTP exchange observed with KRAS G12C.

The compounds were tested in 10 concentration IC₅₀ mode with 3-fold serial dilution at a starting concentration of 10 μM for the Examples and MRTX-849 (reference standard) and 5 μM for ARS-1620 (reference standard). The compound pre-incubation time was 30 min at RT and the curve fits were performed when the activities at the highest concentration of compounds were less than 65%.

### Reaction Buffer:

40 mM HEPES 7.4, 10 mM MgCl₂, 1 mM DTT 0.002% Triton×100, 0.5 DMSO.

### Protein:

SOS1 (RBC cat #MSC-11-502). Recombinant human SOS1 (Genbank accession #NM_005633.3; aa 564-1049, expressed in E. Coli with C-terminal StrepII. MW=60.59 kDa).

### KRAS G12C:

Recombinant human KRAS (Genbank accession #NM_033360.3; aa 2-169, expressed in E. coli with N-terminal TEV cleavable his-tag. MW 21.4 kDa) KRAS is pre-loaded with a 5× excess of Bodipy-GDP. The excess Bodipy-GDP is separated from loaded protein using a spin desalting column.

### Final Concentrations:

KRAS-bodipy-GDP was 0.125 μM; SOS1 was 70 nM; and GTP was 25 μM.

### Reaction Procedure:

Deliver 10 μL of 1.5×KRAS solution in freshly prepared reaction buffer to reaction wells.
1. Deliver compounds in 100% DMSO into buffer using acoustic technology (Echo550; nanoliter range).
2. Incubate compounds for 30 minutes at room temperature.
3. Prepare 3× (SOS1+GTP) solution in reaction buffer.
4. Deliver 5 μL of SOS1+GTP solution into reactions wells (deliver GTP only to column 1 for no SOS1 control).
5. Monitor the reaction progress for 30 minutes at RT by measuring the decrease in fluorescence signal in a PHERAstar plate reader (BMG Labtech; Ex/Em=485/520) with real time reaction monitoring.

### Data Analysis (for Covalent Compounds):

The dRFU value at each compound concentration was calculated by subtracting fluorescence (RFU) at the end of 30-minute reaction from the initial fluorescence measured just prior to addition of SOS1/GTP mixture. The fluorescence data was normalized using the equation below and fitted to “one phase exponential decay” equation using GraphPad prism software. The plateau was unconstrained and the dRFU value was used to calculate the IC₅₀ values.

$$Ynorm = 1 - \frac{( Yraw - Ao )}{( M - Ao )}$$

where Yraw is defined as fluorescence at time t, Ao is the average initial fluorescence with no SOS1, and M is the minimum fluorescence at the end of the reaction at the maximum SOS1.

The background subtracted signals (no SOS1 protein wells were used as background) were converted to % activity relative to DMSO controls. Data was analyzed using GraphPad Prism 4 with “sigmoidal dose-response (variable slope)”; 4 parameters with Hill Slope. The constraints were bottom (constant equal to 0) and top (must be less than 120).

### Results

KRAS G12C*

| Example | IC₅₀ (nM) |
|---|---|
| ARS-1620 | 100 ± 18 (n = 7) |
| MRTX-849 | 28 ± 4 (n = 7) |
| Example 1 (1a) | 63 |

*The substrate was Bodipy-GDP/Kras G12C with 0.5% DMSO added to the reaction. IC₅₀ values were calculated using the dRFU analysis method for covalent inhibitors. ARS-1620 and MRTX-849 are reference standards.

### Pharmacokinetics of Example 1 in Male CD-1 Mice

The pharmacokinetic profile of the Example 1 (compound 1a) was determined in male CD-1 mice by WuXi AppTec Co., Ltd., 1318 Wuzhong Avenue, Wuzhong District, Suzhou, China, 215104. The results shown in the table below clearly indicate that Example 1 has an oral bioavailability of 28.8% in male CD-1 mice.

| | Analyte | |
|---|---|---|
| | Example 1 (compound 1a) | |
| | Group | |
| | 3 mg/kg, IV, 10 | 30 mg/kg, PO, 10 |
| | mL/kg, (20% DMSO/30% | mL/kg, (1% Tween 80/ |
| | PEG400/50% dd H₂O) | 2% HPMC/97% H₂O) |
| Matrix | Mean Plasma | Mean Plasma |
| Dosage (mg/kg) | 3.00 | 30.0 |
| Rsq_adj | 0.828 | 0.991 |
| No. points used | 7.00 | 4.00 |
| for T_{1/2} | | |
| C₀ (ng/mL) | 1274 | — |
| Cₘₐₓ (ng/mL) | — | 1215 |
| Tₘₐₓ (h) | — | 1.00 |
| T_{1/2} (h) | 1.89 | 2.88 |
| Vdₛₛ (L/kg) | 3.44 | — |
| Cl(mL/min/kg) | 20.4 | — |
| Tₗₐₛₜ (h) | 8.00 | 8.00 |
| AUC₀₋ₗₐₛₜ | 2225 | 5860 |
| (ng ·h/mL) | | |
| AUC_{0-inf} | 2451 | 7068 |
| (ng · h/mL) | | |
| MRT₀₋ₗₐₛₜ (h) | 2.00 | 3.22 |
| MRT_{0-inf} (h) | 2.81 | 4.75 |
| AUC_{Extra} (%) | 9.23 | 17.1 |
| AUMC_{Extra} (%) | 35.2 | 43.8 |
| Bioavailability | — | 28.8 |
| (% F) | | |

NOTES:
If the adjusted Rsq (linear regression coefficient of the concentration value on the terminal phase) is less than 0.9, T_{1/2} might not be accurately estimated. If the % AUC_{Extra} > 20%, AUC_{0-inf}, Cl, MRT_{0-inf} and Vdₛₛ might not be accurately estimated. If the % AUMC_{Extra} > 20%, MRT_{0-inf} and Vdₛₛ might not be accurately estimated. Composite mean concentration was used in PK parameter calculation. The adjusted linear regression coefficient of the concentration value on the terminal phase is less than 0.9, T_{1/2} might not be accurately estimated. Composite mean concentration was used in PK parameter calculation. Bioavailability (% F) was calculated by using AUC_{0-inf} (if all AUC_{Extra} < 20%) or AUC0-last (if one or more AUC_{Extra} > 20%) with the Nominal Dose.

1. A compound of Formula I: 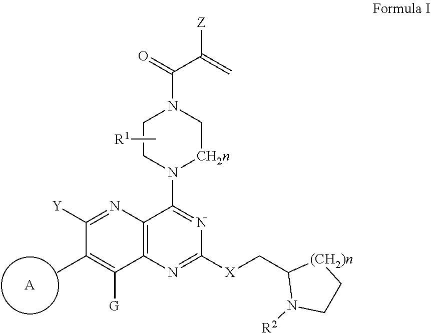
or a salt, solvate, or prodrug thereof, wherein:
A is chosen from aryl or heteroaryl optionally substituted with one or more of hydrogen, halogen, hydroxyl, C₁₋₆alkyl, C₂-C₆alkenyl, C₂-C₆alkynyl, —(C₀-C₆alkyl)cycloalkyl, C₁₋₆haloalkyl, C₁₋₆alkoxy, NO₂, cyano, CO₂H, PO(OR³)₂, POR³(OR³), PO(R⁴)₂, NH₂, NH(C₁₋₆ alkyl) or N(C₁₋₆ alkyl)₂;
X is chosen from O, NR², S or CH₂;
Y and G may be the same or different and chosen from bromine, iodine, C₁₋₄alkyl, C₁₋₄ perdeuteroalkyl, —(C₀-C₂alkyl)alkenyl, —(C₀-C₂alkyl)alkynyl, —(C₀-C₂alkyl)cycloalkyl, C₁₋₄haloalkyl, —O(C₁₋₄alkyl), —S(C₁₋₄alkyl), —(C₀-C₂alkyl)cyano, or —O(C₁₋₄ haloalkyl);
Z is chosen from hydrogen, halogen, trifluoromethyl or C₁₋₆ alkyl;
R¹ is chosen from hydrogen, C₁-C₀alkyl, —(C₁-C₆alkyl)C₁₋₆alkoxy, —C₀-C₆alkyl(cycloalkyl), C₁-C₆haloalkyl, —(C₁-C₆alkyl)CN or —(C₁-C₆alkyl)P(O)R²R³;
n is 1-3;
Each R² is chosen from H, C₁₋₆ alkyl, C₃₋₆cycloalkyl or —(C₁-C₆alkyl)P(O)R²R³;
R³ is chosen from H, C₁₋₆alkyl or C₃₋₆cycloalkyl;
R⁴ is chosen from C₁₋₆ alkyl, C₃₋₆cycloalkyl or aryl.

2. The compound or salt of claim 1 wherein the compound of Formula I is a compound selected from 1b-1z and 2a-2g or a salt, solvate, or prodrug thereof:

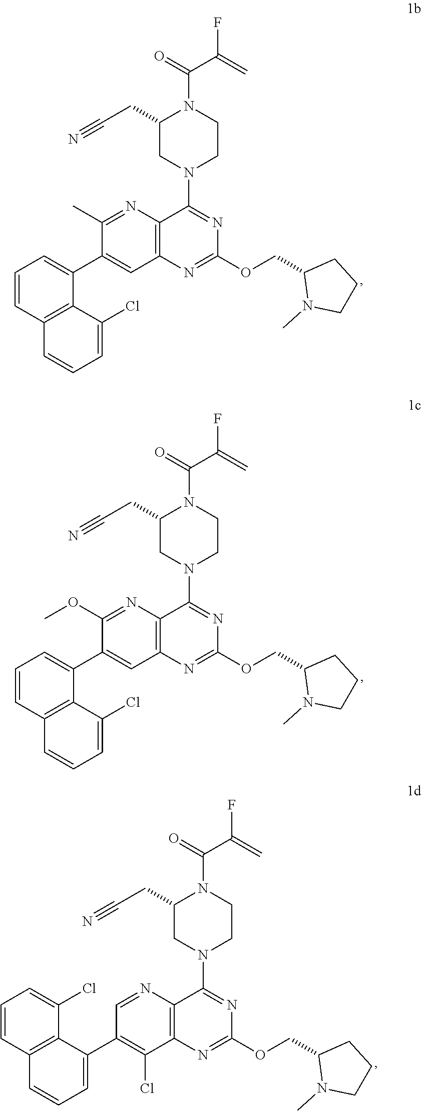

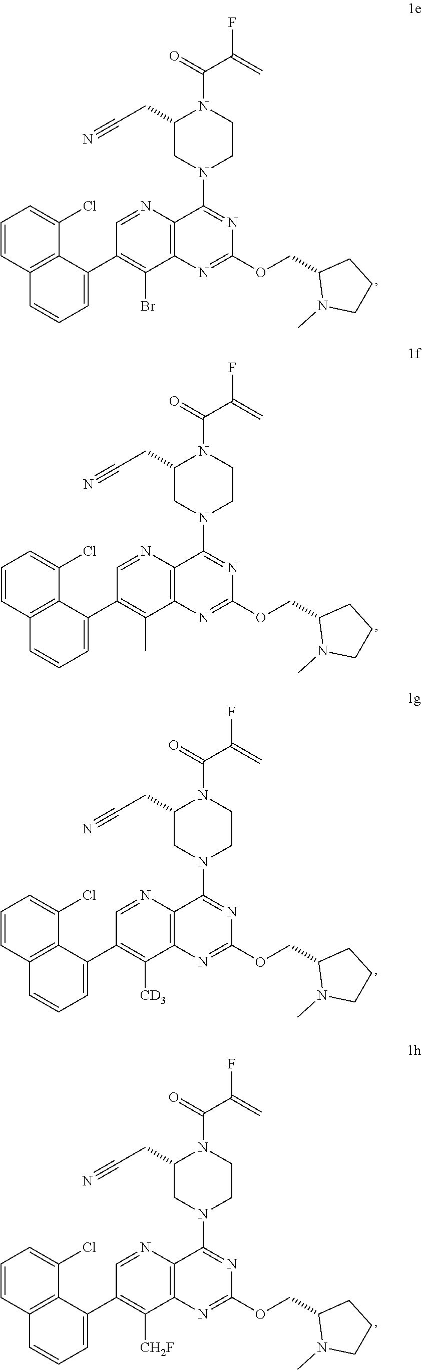

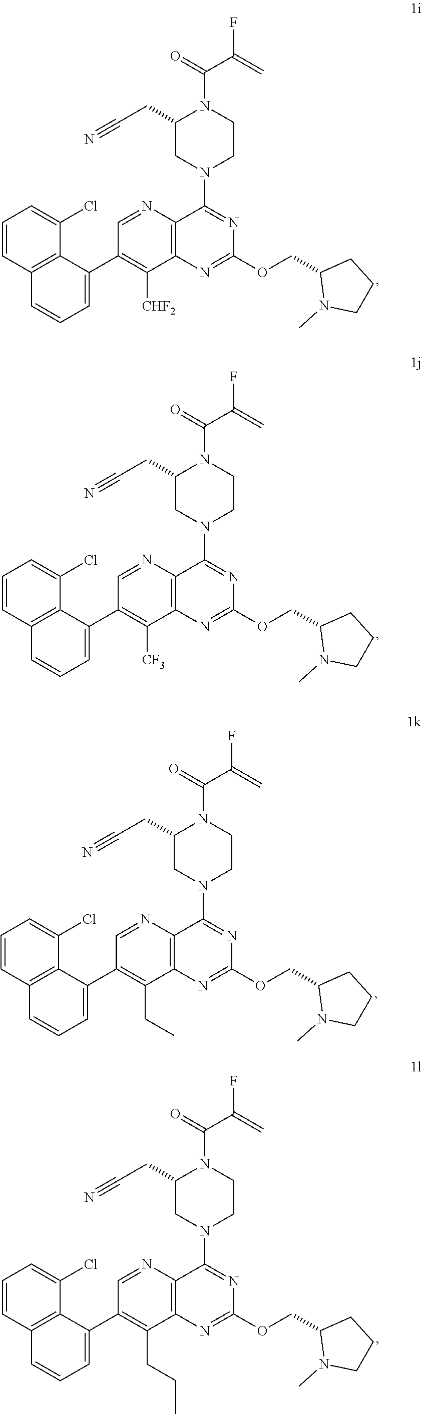

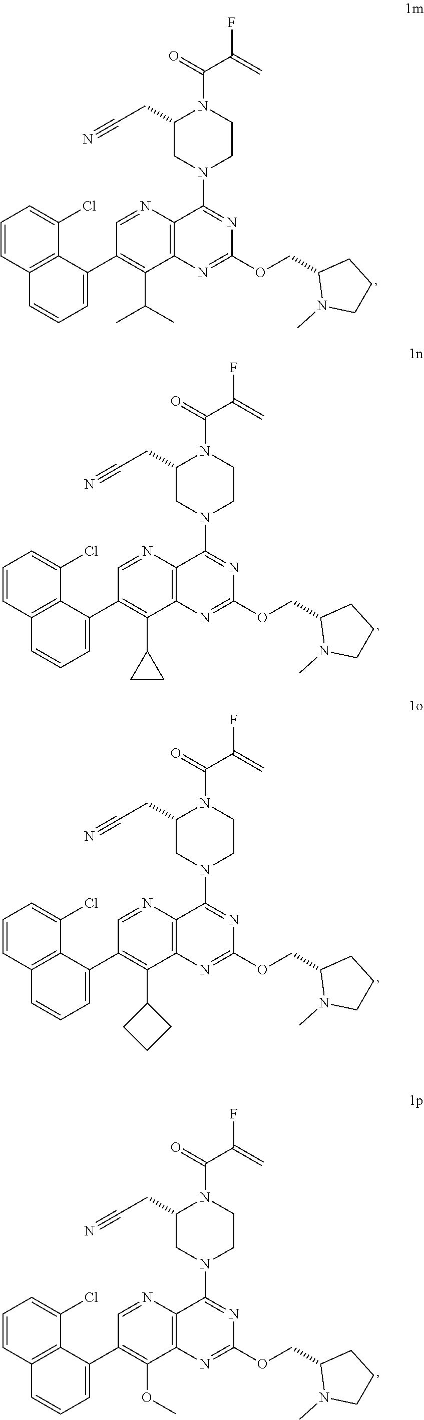

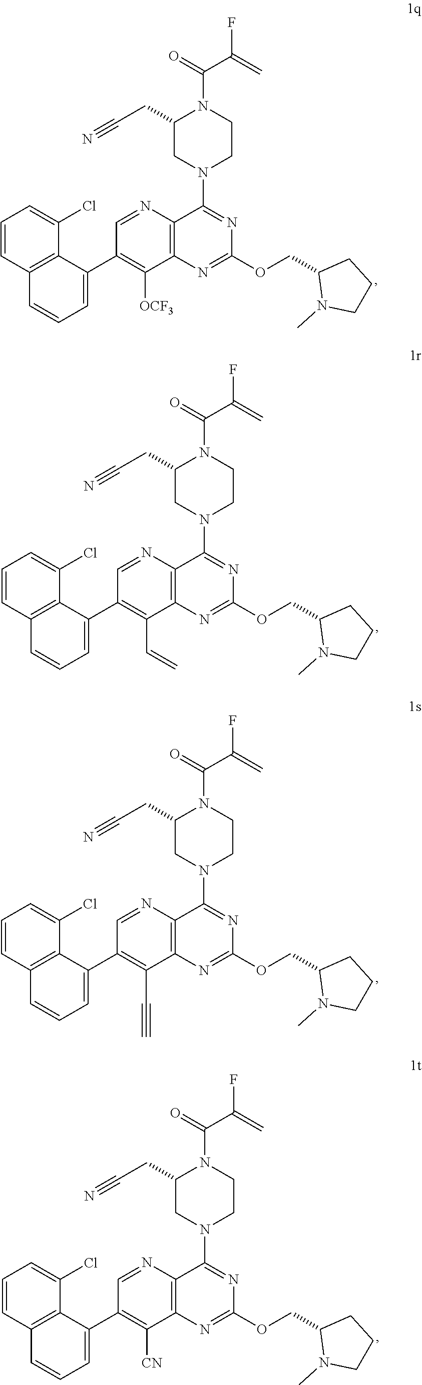

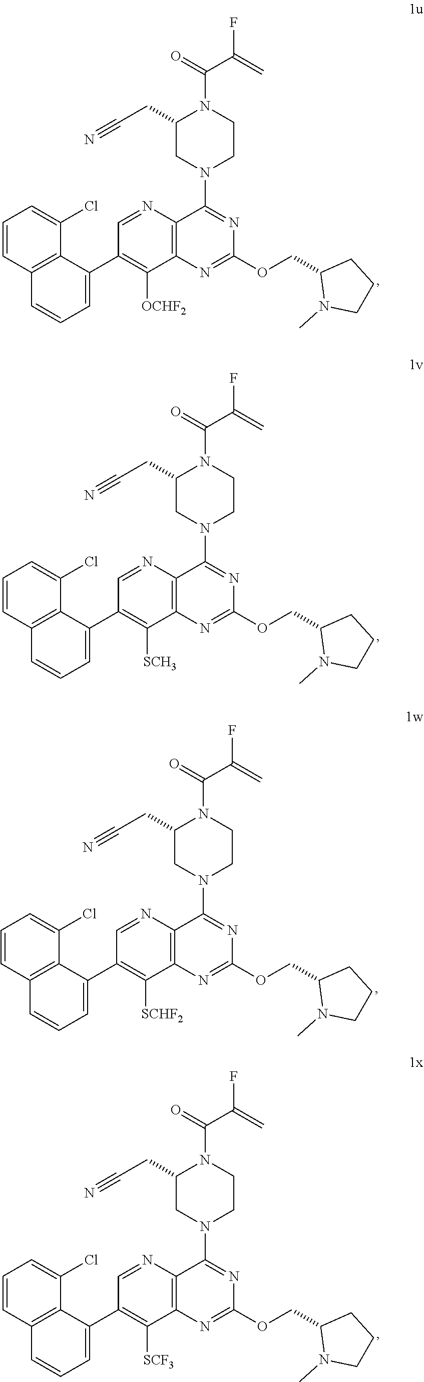

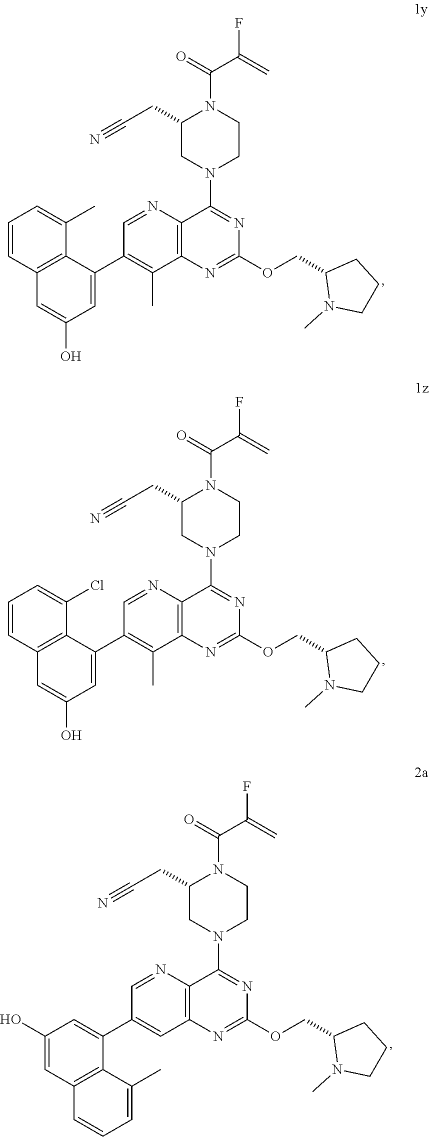

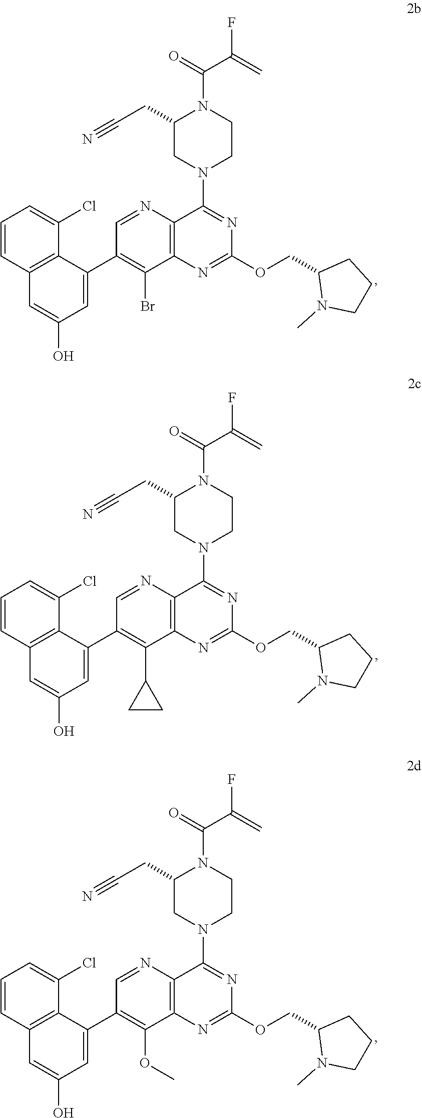

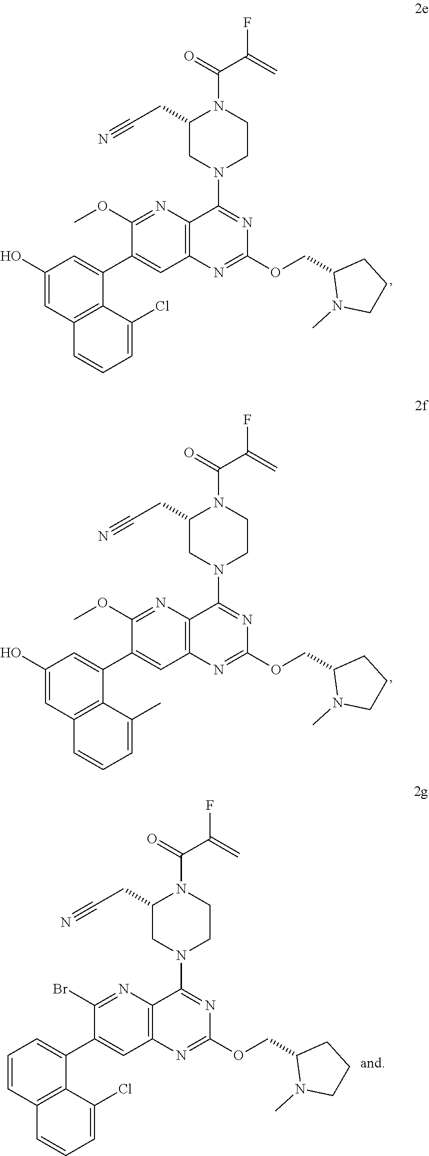

3. A pharmaceutical composition comprising a compound of claim 1, or a salt, solvate, or prodrug thereof, together with a pharmaceutically acceptable carrier.

4. A method of treating a disease, disorder, or medical condition in a patient, comprising the step of providing to a patient in need thereof a therapeutic agent, wherein the therapeutic agent comprises a compound of claim 1, or a salt, solvate or prodrug thereof.

5. The method of claim 4, wherein said disease, disorder or medical condition in a patient comprises various cancers.

6. The method of claim 5, wherein said disease, disorder or medical condition in a patient is mediated through KRAS.

7. The method of claim 6, wherein said disease, disorder, or medical condition is mediated through KRAS, especially the KRAS mutant G12C.

8. The method of claim 6, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

9. The method of claim 4, further comprising administering to the patient in need thereof at least one additional therapeutic agent.

10. The compound of claim 2 wherein the compound of Formula I is a compound selected from compounds 1d, 1f, 1n and 1z or a salt, solvate, or prodrug thereof:

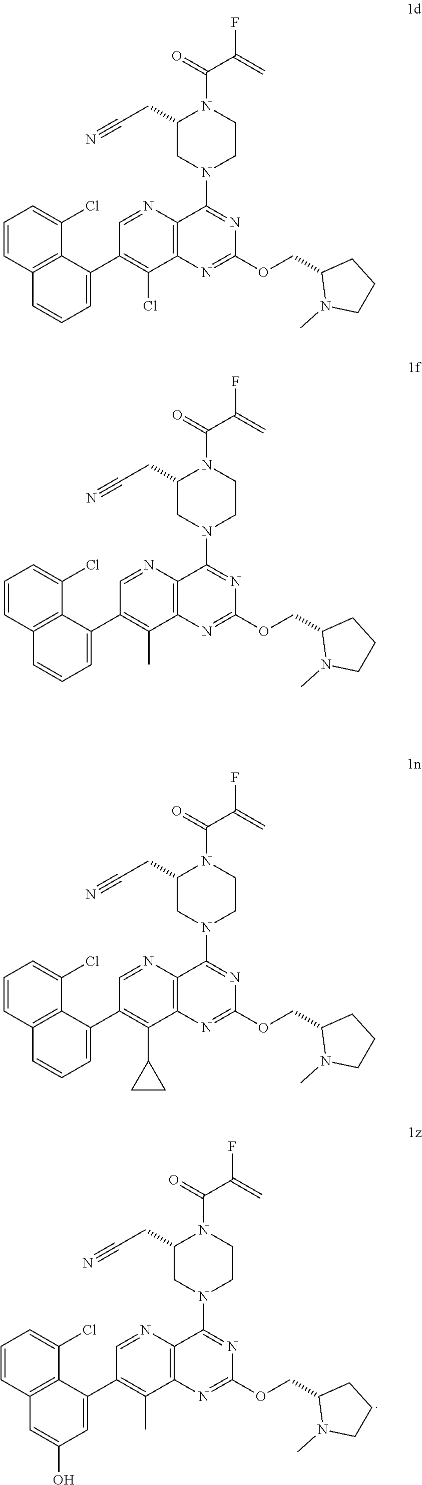

11. The method of claim 4, comprising the step of providing to a patient in need thereof the pharmaceutical composition of claim 3.

12. The method of claim 4, wherein said disease, disorder or medical condition in a patient comprises various cancers.

13. The method of claim 12, wherein said disease, disorder, or medical condition is mediated through KRAS.

14. The method of claim 13, wherein said disease, disorder, or medical condition is mediated through KRAS mutant G12C.

15. The method of claim 13, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.
